SEQUENCE LISTING (10 sequences)

SEQ ID NO: 1 - protein, 116 aa, Artificial Sequence
  Heavy Chain Variable Region
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSS

SEQ ID NO: 2 - protein, 108 aa, Artificial Sequence
  Light Chain Variable Region
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIK

SEQ ID NO: 3 - protein, 8 aa, Artificial Sequence
  Heavy Chain CDR 1
GFTFSNYE

SEQ ID NO: 4 - protein, 8 aa, Artificial Sequence
  Heavy Chain CDR 2
IRTSGTTK

SEQ ID NO: 5 - protein, 9 aa, Artificial Sequence
  Heavy Chain CDR 3
AGGGTFLHY

SEQ ID NO: 6 - protein, 6 aa, Artificial Sequence
  Light Chain CDR 1
QGIASY

SEQ ID NO: 7 - protein, 3 aa, Artificial Sequence
  Light Chain CDR 2
AAS

SEQ ID NO: 8 - protein, 10 aa, Artificial Sequence
  Light Chain CDR 3
QQAKSFPMYT

SEQ ID NO: 9 - protein, 446 aa, Artificial Sequence
  Full-length Heavy Chain
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  Full-length Light Chain
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0031843 A1
(43) Pub. Date: Feb. 3, 2022

(54) Stabilized Formulations Containing Anti-CTLA-4 Antibodies

(71) Applicant: Regeneron Pharmaceuticals, Inc., Tarrytown, NY (US)

(72) Inventors: Yuan CHENG, Livingston, NJ (US); David B. LUDWIG, Saratoga, NY (US); Xiaolin TANG, Old Tappan, NJ (US)

(21) Appl. No.: 17/369,321

(22) Filed: Jul. 7, 2021

Related U.S. Application Data
(60) Provisional application No. 63/049,540, filed on Jul. 8, 2020.

(51) Int. Cl. A61K 39/395; C07K 16/28; A61K 9/08; A61K 9/19; A61K 47/18; A61K 47/26

(52) U.S. Cl. CPC A61K 39/39591; C07K 16/2818; A61K 9/08; C07K 2317/76; A61K 47/183; A61K 47/26; C07K 2317/21; A61K 9/19

(57) ABSTRACT

The present invention provides liquid and lyophilized pharmaceutical formulations comprising an antibody that specifically binds to human cytotoxic T-lymphocyte-associated protein 4 (hCTLA-4). The formulations may contain, in addition to an anti-CTLA-4 antibody, a buffer, at least one sugar, or at least one non-ionic surfactant. The pharmaceutical formulations of the present invention exhibit a substantial degree of antibody stability after storage for several months and after being subjected to thermal and other physical stresses.

### CROSS REFERENCE TO RELATED APPLICATION

This application claims the benefit under 35 USC § 119(e) of US Provisional Application No. 63,049,540, filed Jul. 8, 2020, which is incorporated herein by reference in its entirety for all purposes.

### REFERENCE TO A SEQUENCE LISTING

This application incorporates by reference the Sequence Listing submitted in Computer Readable Form as file 10813US01-Sequence.txt, created on Jul. 7, 2021 and containing 9,333 bytes.

### FIELD OF THE INVENTION

The present invention relates to the field of therapeutic antibody formulations. More specifically, the present invention relates to the field of pharmaceutical formulations comprising a human antibody that specifically binds to human CTLA-4.

### BACKGROUND

Cytotoxic T-lymphocyte-associated protein 4 (CTLA-4; also known as CD152) is a type I transmembrane T cell inhibitory checkpoint receptor expressed on conventional and regulatory T cells. CTLA-4 negatively regulates T cell activation by outcompeting the stimulatory receptor CD28 from binding to its natural ligands, B7-1 (CD80) and B7-2 (CD86). Initial T-cell activation is achieved by stimulating T-cell receptors (TCR) that recognize specific peptides presented by major histocompatibility complex class I or II (MHCI or MHCII) proteins on antigen-presenting cells (APC) (Goldrath et al. 1999, Nature 402: 255-262). An activated TCR in turn initiates a cascade of signaling events, which can be monitored by expression of transfected reporter genes, driven by promoters regulating the expression of various transcription factors such as activator-protein 1 (AP-1), Nuclear Factor of Activated T-cells (NFAT) or Nuclear factor kappa-light-chain-enhancer of activated B cells (NFκB). The T-cell response is then further refined via engagement of co-stimulatory or co-inhibitory receptors expressed either constitutively or inducibly on T-cells such as CD28, CTLA-4 (Cytotoxic T-Lymphocyte-Associated Protein 4), PD-1 (Programmed Cell Death Protein 1), LAG-3 (Lymphocyte-Activation Gene 3) or other molecules (Sharpe et al. 2002, Nat. Rev. Immunol. 2: 116-126).

Therapeutic macromolecules (e.g., antibodies) must be formulated in a manner that not only makes the molecules suitable for administration to patients, but also maintains their stability during storage. For example, therapeutic antibodies are prone to degradation, aggregation and/or undesired chemical modifications unless the solution is formulated properly. The stability of an antibody in formulation depends not only on the kinds of excipients used in the formulation, but also on the amounts and proportions of the excipients relative to one another. Thus, when formulating a therapeutic antibody, great care must be taken to arrive at a formulation that remains stable, contains an adequate concentration of antibody, and possesses other properties which enable the formulation to be conveniently administered to patients.

Antibodies to human CTLA-4 are one example of therapeutically relevant macromolecules that require proper formulation.

Although anti-CTLA-4 antibodies are known in the art (see, e.g., WO 2019/023482), there remains a need for pharmaceutical formulations comprising anti-CTLA-4 antibodies that are sufficiently stable and suitable for administration to patients.

### BRIEF SUMMARY OF THE INVENTION

Stable pharmaceutical formulations comprising an anti-CTLA-4 antibody and one or more excipients, as well as kits comprising such formulations and uses thereof, are provided. In some cases, the pharmaceutical formulations are liquid formulations. In some cases, the pharmaceutical formulations are lyophilized formulations. In some cases, the pharmaceutical formulations are reconstituted formulations from a lyophilized drug product.

In one aspect, the present disclosure provides a stable liquid pharmaceutical formulation comprising, in an aqueous solution: (i) an antibody that specifically binds to human cytotoxic T-lymphocyte-associated protein 4 (hCTLA-4); (ii) a buffer; (iii) a thermal stabilizer; and (iv) an organic cosolvent. In some embodiments, the formulation is a reconstituted formulation (i.e., reconstituted from a lyophilized drug product). In another aspect, the present disclosure provides a stable lyophilized pharmaceutical formulation of an antibody that specifically binds to human cytotoxic T-lymphocyte-associated protein 4 (hCTLA-4), made by lyophilizing an aqueous solution comprising: a buffer; a thermal stabilizer; and an organic cosolvent.

In some embodiments, the buffer is histidine at a concentration of from 1 mM to 30 mM. In some embodiments, the thermal stabilizer is sucrose at a concentration of from 1% w/v to 15% w/v. In some embodiments, the organic cosolvent is a surfactant at a concentration of from 0.01% w/v to 0.3% w/v. In some cases, the surfactant is polysorbate 20.

In some embodiments, the antibody is present at a concentration of from 1 mg/ml to 200 mg/ml in the aqueous solution. In some embodiments, the antibody is present at a concentration of from 25 mg/ml±2.5 mg/ml to 100 mg/ml±10 mg/ml.

In some embodiments, the antibody comprises the complementarity determining regions (HCDR1-HCDR2-HCDR3) of a heavy chain variable region (HCVR) comprising the amino acid sequence of SEQ ID NO: 1, and the complementarity determining regions (LCDR1-LCDR2-LCDR3) of a light chain variable region (LCVR) comprising the amino acid sequence of SEQ ID NO: 2. In some embodiments, the antibody comprises HCDR1-HCDR2-HCDR3 domains comprising the amino acid sequences of SEQ ID NOs: 3-4-5, respectively, and LCDR1-LCDR2-LCDR3 domains comprising the amino acid sequences of SEQ ID NOs: 6-7-8, respectively. In some embodiments, the antibody comprises a heavy chain variable region (HCVR) comprising the amino acid sequence of SEQ ID NO: 1, and a light chain variable region (LCVR) comprising the amino acid sequence of SEQ ID NO: 2.

In some embodiments, the antibody has a human IgG heavy chain constant region. In some cases, the heavy chain constant region is of isotype IgG1. In some cases, the heavy chain constant region is of isotype IgG4.

In some embodiments, the antibody comprises a heavy chain comprising the amino acid sequence of residues 1-445 of SEQ ID NO: 9 and a light chain comprising the amino acid sequence of SEQ ID NO: 10. In some embodiments, the antibody comprises a heavy chain comprising the amino acid sequence of SEQ ID NO: 9 and a light chain comprising the amino acid sequence of SEQ ID NO: 10.

In any of the various embodiments, the aqueous solution comprises: (i) about 5 mg/ml to about 150 mg/mL of the antibody that specifically binds to hCTLA-4; (ii) about 5 mM to about 25 mM histidine; (iii) about 3% w/v to about 12% w/v sucrose; and (iv) about 0.05% w/v to about 0.25% w/v polysorbate 20. In some embodiments, the aqueous solution has a pH of from about 5.5 to about 6.5.

In any of the various embodiments, the aqueous solution comprises: (i) about 50 mg/ml±5 mg/ml of the antibody; (ii) about 10 mM±1 mM histidine; (iii) about 5% w/v±0.5% w/v sucrose; and (iv) about 0.1%±0.01% w/v polysorbate 20. In any of the various embodiments, the aqueous solution comprises: (i) about 100 mg/ml±10 mg/ml of the antibody; (ii) about 20 mM±2 mM histidine; (iii) about 10% w/v±1% w/v sucrose; and (iv) about 0.2%±0.02% w/v polysorbate 20. In some cases, the pH of the aqueous solution is from 5.8 to 6.2. In some cases, the pH of the aqueous solution is about 6.0.

In one aspect, the present disclosure provides a stable liquid pharmaceutical formulation comprising, in an aqueous solution: (i) a human antibody that specifically binds to human cytotoxic T-lymphocyte-associated protein 4 (hCTLA-4) at a concentration of from 5±0.5 mg/ml to 150±15 mg/ml, wherein the antibody comprises a HCVR comprising HCDR1, HCDR2, and HCDR3 domains comprising the amino acid sequences of SEQ ID NOs: 3, 4, and 5, respectively, and LCDR1, LCDR2, and LCDR3 domains comprising the amino acid sequences of SEQ ID NOs: 6, 7, and 8, respectively; (ii) 5 mM to 25 mM histidine; (iii) 3% w/v to 12% w/v sucrose; and (iv) 0.05% w/v to 0.25% w/v polysorbate 20, wherein the aqueous solution has a pH of from 5.8 to 6.2. In some embodiments, the formulation is a reconstituted formulation.

In one aspect, the present disclosure provides a stable lyophilized pharmaceutical formulation of an antibody that specifically binds to human cytotoxic T-lymphocyte-associated protein 4 (hCTLA-4), made by lyophilizing an aqueous solution comprising: (i) a human antibody that specifically binds to human cytotoxic T-lymphocyte-associated protein 4 (hCTLA-4) at a concentration of from 5±0.5 mg/ml to 150±15 mg/ml, wherein the antibody comprises a HCVR comprising HCDR1, HCDR2, and HCDR3 domains comprising the amino acid sequences of SEQ ID NOs: 3, 4, and 5, respectively, and LCDR1, LCDR2, and LCDR3 domains comprising the amino acid sequences of SEQ ID NOs: 6, 7, and 8, respectively; (ii) 5 mM to 25 mM histidine; (iii) 3% w/v to 12% w/v sucrose; and (iv) 0.05% w/v to 0.25% w/v polysorbate 20, wherein the aqueous solution has a pH of from 5.8 to 6.2.

In some embodiments (e.g., the formulations discussed in the preceding two paragraphs), the antibody comprises a heavy chain variable region (HCVR) comprising the amino acid sequence of SEQ ID NO: 1, and a light chain variable region (LCVR) comprising the amino acid sequence of SEQ ID NO: 2. In some embodiments, the antibody has a human IgG heavy chain constant region. In some cases, the heavy chain constant region is of isotype IgG1. In some cases, the heavy chain constant region is of isotype IgG4.

In one aspect, the present disclosure provides a stable liquid pharmaceutical formulation comprising, in an aqueous solution: (i) a human antibody that specifically binds to human cytotoxic T-lymphocyte-associated protein 4 (hCTLA-4) at a concentration of from 5±0.5 mg/ml to 150±15 mg/ml, wherein the antibody comprises a heavy chain comprising the amino acid sequence of SEQ ID NO: 9, and a light chain comprising the amino acid sequence of SEQ ID NO: 10; (ii) 5 mM to 25 mM histidine; (iii) 3% w/v to 12% w/v sucrose; and (iv) 0.05% w/v to 0.25% w/v polysorbate 20, wherein the aqueous solution has a pH of from 5.8 to 6.2. In some embodiments, the formulation is a reconstituted formulation.

In one aspect, the present disclosure provides a stable lyophilized pharmaceutical formulation of an antibody that specifically binds to human cytotoxic T-lymphocyte-associated protein 4 (hCTLA-4), made by lyophilizing an aqueous solution comprising: (i) a human antibody that specifically binds to human cytotoxic T-lymphocyte-associated protein 4 (hCTLA-4) at a concentration of from 5±0.5 mg/ml to 150±15 mg/ml, wherein the antibody comprises a heavy chain comprising the amino acid sequence of SEQ ID NO: 9, and a light chain comprising the amino acid sequence of SEQ ID NO: 10; (ii) 5 mM to 25 mM histidine; (iii) 3% w/v to 12% w/v sucrose; and (iv) 0.05% w/v to 0.25% w/v polysorbate 20, wherein the aqueous solution has a pH of from 5.8 to 6.2.

In some embodiments (e.g., the formulations discussed in the preceding two paragraphs), the aqueous solution comprises: (i) about 50 mg/ml±5 mg/ml of the antibody; (ii) about 10 mM±1 mM histidine; (iii) about 5% w/v±0.5% w/v sucrose; and (iv) about 0.1%±0.01% w/v polysorbate 20. In some embodiments (e.g., the formulations discussed in the preceding two paragraphs), the aqueous solution comprises: (i) about 100 mg/ml±10 mg/ml of the antibody; (ii) about 20 mM±2 mM histidine; (iii) about 10% w/v±1% w/v sucrose; and (iv) about 0.2%±0.02% w/v polysorbate 20. In some cases, the pH of the aqueous solution is about 6.0.

In any of the various embodiments of the stable pharmaceutical formulations discussed above or herein, at least 90% of the native form of the antibody is recovered after 24 months of storage at 5° C., as determined by size exclusion-ultra performance liquid chromatography (SE-UPLC). In some cases, at least 95% of the native form of the antibody is recovered after 24 months of storage at 5° C., as determined by SE-UPLC. In some cases, at least 97% of the native form of the antibody is recovered after 24 months of storage at 5° C., as determined by SE-UPLC.

In any of the various embodiments of the stable pharmaceutical formulations discussed above or herein, at least 90% of the native form of the antibody is recovered after six months of storage at 25° C. and 60% relative humidity, as determined by SE-UPLC. In some cases, at least 96% of the native form of the antibody is recovered after six months of storage at 25° C. and 60% relative humidity, as determined by SE-UPLC.

In any of the various embodiments of the stable pharmaceutical formulations discussed above or herein, at least 90% of the native form of the antibody is recovered after one month of storage at 45° C. or 50° C., as determined by SE-UPLC. In some cases, at least 95% of the native form of the antibody is recovered after one month of storage at 45° C. or 50° C., as determined by SE-UPLC.

In any of the various embodiments of the stable pharmaceutical formulations discussed above or herein, at least 90% of the native form of the antibody is recovered after sixty minutes of agitation at ambient temperature, as determined by SE-UPLC. In some cases, at least 96% of the native form of the antibody is recovered after sixty minutes of agitation at ambient temperature, as determined by SE-UPLC.

In any of the various embodiments of the stable pharmaceutical formulations discussed above or herein, the formulation comprises no more than 2% high molecular weight (HMW) species after 24 months of storage at 5° C., as determined by SE-UPLC.

In any of the various embodiments of the stable pharmaceutical formulations discussed above or herein, the formulation comprises no more than 2.5% HMW species after six months of storage at 25° C. and 60% relative humidity, as determined by SE-UPLC.

In any of the various embodiments of the stable pharmaceutical formulations discussed above or herein, the formulation comprises no more than 2% high molecular weight (HMW) species after one month of storage at 45° C., as determined by SE-UPLC.

In any of the various embodiments of the stable pharmaceutical formulations discussed above or herein, the formulation comprises no more than 3% high molecular weight (HMW) species after one month of storage at 50° C., as determined by SE-UPLC.

In any of the various embodiments of the stable pharmaceutical formulations discussed above or herein, the formulation comprises no more than 2% high molecular weight (HMW) species after sixty minutes of agitation at ambient temperature, as determined by SE-UPLC.

In some embodiments, the stable pharmaceutical formulation discussed above or herein is contained in a glass vial. In some embodiments, the stable pharmaceutical formulation discussed above or herein is contained in a syringe. In some cases, the syringe comprises a fluorocarbon-coated plunger, or the syringe is a low tungsten syringe. In some case, the syringe is a prefilled syringe. In some cases, the syringe is a prefilled staked needle syringe. In some embodiments, the stable pharmaceutical formulation discussed above or herein is contained in a large volume device or bolus injector.

In one aspect, the present disclosure provides a pen or autoinjector delivery device containing a stable pharmaceutical formulation as discussed above or herein. In some cases, the delivery device is a disposable pen delivery device. In some cases, the delivery device is a reusable pen delivery device.

In one aspect, the present disclosure provides a container containing a stable pharmaceutical formulation as discussed above or herein.

In one aspect, the present disclosure provides a kit comprising (i) a container containing the stable pharmaceutical formulation as discussed above or herein, and (ii) labeling for use of the pharmaceutical formulation. In some embodiments, the labeling recites subcutaneous administration of the pharmaceutical formulation. In some embodiments, the labeling recites intravenous administration of the pharmaceutical formulation.

In one aspect, the present disclosure provides a unit dosage form comprising a stable pharmaceutical formulation as discussed above or herein, wherein the anti-CTLA-4 antibody is present in an amount of from 1 mg to 500 mg. In some cases, the formulation is contained in a glass vial. In some cases, the formulation is contained in a syringe. In some embodiments, the syringe is a prefilled syringe.

In one aspect, the present disclosure provides a safety system delivery device containing a stable pharmaceutical formulation as discussed above or herein. In some embodiments, the safety system delivery device includes a safety sleeve configured to extend by manual operation. In some embodiments, the safety system delivery device includes a safety sleeve configured to automatically extend following injection of the stable pharmaceutical formulation.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure. Each of the values discussed above or herein may be expressed with a variation of 1%, 5%, 10% or 20%. For example, a concentration of 10 mM may be expressed as 10 mM±0.1 mM (1% variation), 10 mM±0.5 mM (5% variation), 10 mM±1 mM (10% variation) or 10 mM±2 mM (20% variation).

Other embodiments will become apparent from a review of the detailed description.

### BRIEF DESCRIPTION OF THE DRAWING

FIG. 1 illustrates lyophilization parameters, including shelf temperature, product temperature, and chamber pressure, of an exemplary drug product lyophilization cycle for production of a lyophilized formulation of an anti-CTLA-4 antibody (mAb1) in accordance with an embodiment of the present disclosure.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.).

All patents, applications and non-patent publications mentioned in this specification are incorporated herein by reference in their entireties.

### Pharmaceutical Formulations

As used herein, the expression “pharmaceutical formulation” means a combination of at least one active ingredient (e.g., an anti-CTLA-4 antibody, etc. which is capable of exerting a biological effect in a human or non-human animal), and at least one inactive ingredient which, when combined with the active ingredient and/or one or more additional inactive ingredients, is suitable for therapeutic administration to a human or non-human animal. The term “formulation,” as used herein, means “pharmaceutical formulation” unless specifically indicated otherwise. The present invention provides pharmaceutical formulations comprising at least one therapeutic polypeptide. According to certain embodiments of the present invention, the therapeutic polypeptide is an antibody that binds specifically to human cytotoxic T-lymphocyte-associated protein 4 (CTLA-4) or an antigen-binding fragment thereof. More specifically, the present invention includes pharmaceutical formulations that comprise: (i) a human antibody that specifically binds to CTLA-4; (ii) a buffer; (iii) a thermal stabilizer; and (iv) a surfactant (also organic cosolvent or interfacial stabilizer). Additional components may be included in the formulations of the present invention if such components do not significantly interfere with the stability of the formulation. Specific exemplary components and formulations included within the present invention are described in detail below.

The pharmaceutical formulations of the present invention may, in certain embodiments, be fluid formulations. As used herein, the expression “fluid formulation” means a mixture of at least two components that exists predominantly in the fluid state at about 2° C. to about 45° C. Fluid formulations include, inter alia, liquid formulations and reconstituted lyophilized formulations. Fluid formulations may be of low, moderate or high viscosity depending on their particular constituents. The pharmaceutical formulations of the present invention may, in certain embodiments, be lyophilized formulations. The terms “lyophilization,” “lyophilized,” and “freeze-dried” refer to a process by which the material to be dried is first frozen and then the ice or frozen solvent is removed by sublimation in a vacuum environment. An excipient may be included in a pre-lyophilized formulation to enhance stability of the lyophilized product upon storage, or to enhance stability of the reconstituted product. A “reconstituted” formulation or product is one that has been prepared by dissolving a lyophilized formulation in a diluent such that the protein (e.g., antibody) present in the lyophilized formulation is dispersed in the reconstituted formulation so that the reconstituted formulation is suitable for parenteral administration (e.g., intravenous or subcutaneous administration). “Reconstitution time” is the time that is required to rehydrate a lyophilized formulation with a solution to a substantially particle-free clarified solution. Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g., phosphate-buffered saline), sterile saline solution, Ringer's solution or dextrose solution.

Antibodies that Specifically Bind Human CTLA-4

The pharmaceutical formulations of the present invention may comprise an antibody (e.g., a human antibody), or an antigen-binding fragment thereof, that binds specifically to hCTLA-4. As used herein, the term “hCTLA-4” refers to a human CTLA-4 protein.

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In certain embodiments of the invention, the anti-CTLA-4 antibodies of the invention are human antibodies. The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences. In various embodiments, the anti-CTLA-4 antibody is a human IgG antibody. In various embodiments, the anti-CTLA-4 antibody is a human antibody of isotype IgG1, IgG2, IgG3 or IgG4, or mixed isotype. In some embodiments, the anti-CTLA-4 antibody is a human IgG1 antibody. In some embodiments, the anti-CTLA-4 antibody is a human IgG4 antibody. In any of the embodiments discussed above or herein, the anti-CTLA-4 antibody may comprise a human kappa light chain. In any of the embodiments discussed above or herein, the anti-CTLA-4 antibody may comprise a human lambda light chain.

The antibodies of the invention may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The terms “antigen-binding portion” or “antigen-binding fragment” of an antibody (or simply “antibody portion” or “antibody fragment”), as used herein, refer to one or more fragments of an antibody that retain the ability to specifically bind to hCTLA-4.

An “isolated antibody,” as used herein, is intended to refer to an antibody that is substantially free of other antibodies having different antigenic specificities (e.g., an isolated antibody that specifically binds hCTLA-4 is substantially free of antibodies that specifically bind antigens other than hCTLA-4).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hCTLA-4 may, however, have cross-reactivity to other antigens, such as CTLA-4 molecules from other species (orthologs). In the context of the present invention, multispecific (e.g., bispecific) antibodies that bind to hCTLA-4 as well as one or more additional antigens are deemed to “specifically bind” hCTLA-4. Moreover, an isolated antibody may be substantially free of other cellular material and/or chemicals.

Exemplary anti-hCTLA-4 antibodies that may be included in the pharmaceutical formulations of the present invention are set forth in WO 2019/023482, the disclosure of which is incorporated by reference in its entirety.

According to certain embodiments of the present invention, the anti-hCTLA-4 antibody, or antigen-binding fragment thereof, comprises heavy chain complementarity determining regions HCDR1-HCDR2-HCDR3, respectively, comprising the amino acid sequences of SEQ ID NOs: 3-4-5. According to certain embodiments of the present invention, the anti-hCTLA-4 antibody, or antigen-binding fragment thereof, comprises light chain complementarity determining regions LCDR1-LCDR2-LCDR3, respectively, comprising the amino acid sequences of SEQ ID NOs: 6-7-8.

In certain embodiments, the anti-hCTLA-4 antibody, or antigen-binding fragment thereof, comprises a heavy chain variable region (HCVR) comprising the amino acid sequence of SEQ ID NO: 1. In certain embodiments, the anti-hCTLA-4 antibody, or antigen-binding fragment thereof, comprises a light chain variable region (LCVR) comprising the amino acid sequence of SEQ ID NO: 2. In certain embodiments, the anti-hCTLA-4 antibody, or antigen-binding fragment thereof, comprises a HCVR/LCVR amino acid sequence pair comprising the amino acid sequences of SEQ ID NOs: 1/2. In some embodiments, the anti-CTLA-4 antibody comprises a HCVR/LCVR comprising the amino acid sequences of SEQ ID NOs: 1/2, respectively, and a human IgG1 heavy chain constant region. In some embodiments, the anti-CTLA-4 antibody comprises a HCVR/LCVR comprising the amino acid sequences of SEQ ID NOs: 1/2, respectively, and a human IgG4 heavy chain constant region. In some embodiments, the anti-CTLA-4 antibody comprises a HCVR/LCVR comprising the amino acid sequences of SEQ ID NOs: 1/2, respectively, and a human IgG heavy chain constant region. In some embodiments, the anti-CTLA-4 antibody comprises a HCVR/LCVR comprising the amino acid sequences of SEQ ID NOs: 1/2, respectively, and a human IgG1 or IgG4 heavy chain constant region. In some embodiments, the anti-CTLA-4 antibody comprises a heavy chain comprising the amino acid sequence of SEQ ID NO: 9 and a light chain comprising the amino acid sequence of SEQ ID NO: 10. An anti-CTLA-4 antibody with a HCVR comprising the amino acid sequence of SEQ ID NO: 1 and a LCVR comprising the amino acid sequence of SEQ ID NO: 2 is referred to herein as mAb1. This antibody has a heavy chain comprising the amino acid sequence of SEQ ID NO: 9 and a light chain comprising the amino acid sequence of SEQ ID NO: 10.

The amount of antibody, or antigen-binding fragment thereof, contained within the pharmaceutical formulations of the present invention may vary depending on the specific properties desired of the formulations, as well as the particular circumstances and purposes for which the formulations are intended to be used. In certain embodiments, the pharmaceutical formulations may contain about 1 mg/mL to about 500 mg/mL of antibody; about 5 mg/mL to about 400 mg/mL of antibody; about 5 mg/mL to about 200 mg/mL of antibody; about 15 mg/mL to about 150 mg/mL; about 25 mg/mL to about 180 mg/mL of antibody; about 25 mg/mL to about 150 mg/mL of antibody; about 50 mg/mL to about 100 mg/mL; about 25 mg/mL to about 75 mg/mL; or about 75 mg/mL to about 125 mg/mL of antibody. For example, the formulations of the present invention may by formulations that comprise about 1 mg/mL; about 2 mg/mL; about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 131 mg/mL; about 132 mg/mL; about 133 mg/mL; about 134 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof, that binds specifically to hCTLA-4. In certain embodiments, the pharmaceutical formulations are formulations that may contain 5±0.75 mg/mL to 150±22.5 mg/mL of antibody; 7.5±1.125 mg/mL to 140±21 mg/mL of antibody; 10±1.5 mg/mL to 130±19.5 mg/mL of antibody; 12.5±1.875 mg/mL to 120±18 mg/mL of antibody; 15±2.25 mg/mL to 110±16.5 mg/mL of antibody; 17.5±2.625 mg/mL to 100±15 mg/mL of antibody; 20±3 mg/mL to 90±13.5 mg/mL of antibody; 22.5±3.375 mg/mL to 80±12 mg/mL of antibody; 25±3.75 mg/mL to 70±10.5 mg/mL of antibody; 27.5±4.125 mg/mL to 60±9 mg/mL of antibody; 30±4.5 mg/mL to 50±7.5 mg/mL of antibody; 25±3.75 mg/mL of antibody, 50±7.5 mg/ml of antibody, or 100±15 mg/ml. In some embodiments, the pharmaceutical formulations contain from 15±0.15 mg/ml to 150±1.5 mg/ml of the anti-CTLA-4 antibody. In some cases, the pharmaceutical formulations contain 50 mg/mL±2.5 mg/mL of the anti-CTLA-4 antibody. In some cases, the pharmaceutical formulations contain 100 mg/mL±5 mg/mL of the anti-CTLA-4 antibody.

### Bioequivalents

The present invention encompasses antibodies having amino acid sequences that vary from those of the exemplary molecules disclosed herein but that retain the ability to bind hCTLA-4. Such variant molecules may comprise one or more additions, deletions, or substitutions of amino acids when compared to parent sequence, but exhibit biological activity that is essentially equivalent to that of the antibodies discussed herein.

The present invention includes antigen-binding molecules that are bioequivalent to any of the exemplary antibodies set forth herein. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

### Formulation Excipients and pH

The pharmaceutical formulations of the present invention comprise one or more excipients. The term “excipient,” as used herein, means any non-therapeutic agent added to the formulation to provide a desired consistency, viscosity or stabilizing effect.

In certain embodiments, the pharmaceutical formulations of the present invention may comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some embodiments, the sugar is sucrose. In some cases, the sugar (e.g., sucrose) acts as a thermal stabilizer for the anti-CTLA-4 antibody.

The amount of sugar (e.g., sucrose) contained within the pharmaceutical formulations of the present invention will vary depending on the specific circumstances and intended purposes for which the formulations are used. In certain embodiments, the formulations may contain about 0.1% to about 20% sugar; about 0.5% to about 20% sugar; about 1% to about 20% sugar; about 2% to about 15% sugar; about 3% to about 10% sugar; about 3% to about 7% sugar; about 4% to about 6% sugar, about 3% to about 12% sugar, or about 4% to about 11% sugar. For example, the pharmaceutical formulations of the present invention may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 15%; or about 20% sugar (e.g., sucrose). In some embodiments, the formulations contain about 5% sugar (e.g., sucrose). In some embodiments, the formulations contain about 10% sugar (e.g., sucrose).

The pharmaceutical formulations of the present invention may also comprise one or more organic cosolvents (or interfacial stabilizer) in a type and in an amount that stabilizes the anti-CTLA-4 antibody under conditions of rough handling or agitation, such as, e.g., orbital shaking. In some embodiments, the organic cosolvent is a surfactant. As used herein, the term “surfactant” means a substance which reduces the surface tension of a fluid in which it is dissolved and/or reduces the interfacial tension between oil and water. Surfactants can be ionic or non-ionic. Exemplary non-ionic surfactants that can be included in the formulations of the present invention include, e.g., alkyl poly(ethylene oxide), alkyl polyglucosides (e.g., octyl glucoside and decyl maltoside), fatty alcohols such as cetyl alcohol and oleyl alcohol, cocamide MEA, cocamide DEA, and cocamide TEA. Specific non-ionic surfactants that can be included in the formulations of the present invention include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 188 (also known as Pluronic F68), poloxamer 407; polyethylene-polypropylene glycol; or polyethylene glycol (PEG). Polysorbate 20 is also known as TWEEN 20, sorbitan monolaurate and polyoxyethylenesorbitan monolaurate. In some embodiments, the surfactant is polysorbate 20.

The amount of surfactant contained within the pharmaceutical formulations of the present invention may vary depending on the specific properties desired of the formulations, as well as the particular circumstances and purposes for which the formulations are intended to be used. In certain embodiments, the formulations may contain about 0.01% to about 0.5% surfactant; about 0.05% to about 0.3% surfactant; about 0.04% to about 0.25%; about 0.05% to about 0.24% surfactant; about 0.06% to about 0.23% surfactant; or about 0.07% to about 0.22% surfactant. For example, the formulations of the present invention may comprise about 0.05%; about 0.06%; about 0.07%; about 0.08%; about 0.09%; about 0.10%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; or about 0.30% surfactant (e.g., polysorbate 20). In some embodiments, the formulations contain about 0.1% surfactant (e.g., polysorbate 20). In some embodiments, the formulations contain about 0.2% surfactant (e.g., polysorbate 20). Each of the percentages noted above corresponds to a percent weight/volume (w/v).

The pharmaceutical formulations of the present invention may also comprise a buffer or buffer system, which serves to maintain a stable pH and to help stabilize the anti-CTLA-4 antibody. In some embodiments, the buffer or buffer system comprises at least one buffer that has a buffering range that overlaps fully or in part the range of pH 5.6 to 6.4. In certain embodiments, the buffer comprises a histidine buffer. In certain embodiments, the buffer (e.g., histidine) is present at a concentration of from about 1 mM to about 40 mM, about 1 mM to about 30 mM, about 1 mM to about 20 mM; about 3 mM to about 18 mM; about 5 mM to about 15 mM; or about 8 mM to about 12 mM, about 10 mM to about 20 mM, about 15 mM to about 25 mM, or about 18 mM to about 22 mM. In some embodiments, the buffer (e.g., histidine) is present at a concentration of 10 mM±1 mM, 10 mM±0.5 mM, 10 mM±0.1 mM, 20 mM±2 mM, 20 mM±1 mM, or 20 mM±0.5 mM. In some embodiments, the buffer is present at a concentration of about 5 mM; about 6 mM; about 7 mM; about 8 mM; about 9 mM; about 10 mM; about 11 mM; about 12 mM; about 13 mM; about 14 mM; about 15 mM; about 16 mM; about 17 mM; about 18 mM; about 19 mM; about 20 mM; about 21 mM; about 22 mM; about 23 mM; about 24 mM; or about 25 mM.

### Exemplary Formulations

According to one aspect of the present invention, the pharmaceutical formulation comprises, in an aqueous solution: (i) a human antibody that specifically binds to hCTLA-4 (e.g., mAb1); (ii) a buffer (e.g., histidine); (iii) a thermal stabilizer (e.g., sucrose); and (iv) an organic cosolvent (e.g., polysorbate 20).

In some cases, the stable pharmaceutical formulation comprises, in an aqueous solution, (i) a human antibody that specifically binds to hCTLA-4 (e.g., mAb1) at a concentration of from about 1 mg/ml to about 200 mg/ml; (ii) a buffer (e.g., histidine) at a concentration of from about 5 mM to about 25 mM; (iii) a thermal stabilizer (e.g., sucrose) at a concentration of from about 5% w/v to about 15% w/v; and (iv) an organic cosolvent (e.g., polysorbate 20) at a concentration of from about 0.05% w/v to about 0.25% w/v.

In some cases, the stable pharmaceutical formulation comprises, in an aqueous solution, (i) a human antibody that specifically binds to hCTLA-4 (e.g., mAb1) at a concentration of from about 25 mg/ml to about 100 mg/ml; (ii) a buffer (e.g., histidine) at a concentration of from about 5 mM to about 25 mM; (iii) a thermal stabilizer (e.g., sucrose) at a concentration of from about 5% w/v to about 15% w/v; and (iv) an organic cosolvent (e.g., polysorbate 20) at a concentration of from about 0.05% w/v to about 0.25% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) a buffer (e.g., histidine) at a concentration of from about 5 mM to about 25 mM; (iii) a thermal stabilizer (e.g., sucrose) at a concentration of from about 5% w/v to about 15% w/v; and (iv) an organic cosolvent (e.g., polysorbate 20) at a concentration of from about 0.05% w/v to about 0.25% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG1; (ii) a buffer (e.g., histidine) at a concentration of from about 5 mM to about 25 mM; (iii) a thermal stabilizer (e.g., sucrose) at a concentration of from about 5% w/v to about 15% w/v; and (iv) an organic cosolvent (e.g., polysorbate 20) at a concentration of from about 0.05% w/v to about 0.25% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG4; (ii) a buffer (e.g., histidine) at a concentration of from about 5 mM to about 25 mM; (iii) a thermal stabilizer (e.g., sucrose) at a concentration of from about 5% w/v to about 15% w/v; and (iv) an organic cosolvent (e.g., polysorbate 20) at a concentration of from about 0.05% w/v to about 0.25% w/v.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of from about 25 mg/ml to about 100 mg/ml; (ii) a buffer (e.g., histidine) at a concentration of from about 5 mM to about 25 mM; (iii) a thermal stabilizer (e.g., sucrose) at a concentration of from about 5% w/v to about 15% w/v; and (iv) an organic cosolvent (e.g., polysorbate 20) at a concentration of from about 0.05% w/v to about 0.25% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) histidine at a concentration of from about 8 mM to about 12 mM; (iii) sucrose at a concentration of from about 3% w/v to about 7% w/v; and (iv) polysorbate 20 at a concentration of from about 0.08% w/v to about 0.12% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG1; (ii) histidine at a concentration of from about 8 mM to about 12 mM; (iii) sucrose at a concentration of from about 3% w/v to about 7% w/v; and (iv) polysorbate 20 at a concentration of from about 0.08% w/v to about 0.12% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG4; (ii) histidine at a concentration of from about 8 mM to about 12 mM; (iii) sucrose at a concentration of from about 3% w/v to about 7% w/v; and (iv) polysorbate 20 at a concentration of from about 0.08% w/v to about 0.12% w/v.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of from about 25 mg/ml to about 100 mg/ml; (ii) histidine at a concentration of from about 8 mM to about 12 mM; (iii) sucrose at a concentration of from about 3% w/v to about 7% w/v; and (iv) polysorbate 20 at a concentration of from about 0.08% w/v to about 0.12% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) histidine at a concentration of from about 18 mM to about 22 mM; (iii) sucrose at a concentration of from about 8% w/v to about 12% w/v; and (iv) polysorbate 20 at a concentration of from about 0.18% w/v to about 0.22% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG1; (ii) histidine at a concentration of from about 18 mM to about 22 mM; (iii) sucrose at a concentration of from about 8% w/v to about 12% w/v; and (iv) polysorbate 20 at a concentration of from about 0.18% w/v to about 0.22% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG4; (ii) histidine at a concentration of from about 18 mM to about 22 mM; (iii) sucrose at a concentration of from about 8% w/v to about 12% w/v; and (iv) polysorbate 20 at a concentration of from about 0.18% w/v to about 0.22% w/v.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of from about 25 mg/ml to about 100 mg/ml; (ii) histidine at a concentration of from about 18 mM to about 22 mM; (iii) sucrose at a concentration of from about 8% w/v to about 12% w/v; and (iv) polysorbate 20 at a concentration of from about 0.18% w/v to about 0.22% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) about 10 mM±1 mM histidine; (iii) about 5% w/v±0.5% w/v sucrose; and (iv) about 0.1%±0.01% w/v polysorbate 20.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG1; (ii) about 10 mM±1 mM histidine; (iii) about 5% w/v±0.5% w/v sucrose; and (iv) about 0.1%±0.01% w/v polysorbate 20.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG4; (ii) about 10 mM±1 mM histidine; (iii) about 5% w/v±0.5% w/v sucrose; and (iv) about 0.1%±0.01% w/v polysorbate 20.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of from about 25 mg/ml to about 100 mg/ml; (ii) about 10 mM±1 mM histidine; (iii) about 5% w/v±0.5% w/v sucrose; and (iv) about 0.1%±0.01% w/v polysorbate 20.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) histidine at a concentration of about 20 mM±2 mM; (iii) sucrose at a concentration of about 10% w/v±1% w/v; and (iv) polysorbate 20 at a concentration of about 0.2% w/v±0.02% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG1; (ii) histidine at a concentration of about 20 mM±2 mM; (iii) sucrose at a concentration of about 10% w/v±1% w/v; and (iv) polysorbate 20 at a concentration of about 0.2% w/v±0.02% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml, wherein the antibody has a heavy chain constant region of isotype IgG4; (ii) histidine at a concentration of about 20 mM±2 mM; (iii) sucrose at a concentration of about 10% w/v±1% w/v; and (iv) polysorbate 20 at a concentration of about 0.2% w/v±0.02% w/v.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of from about 25 mg/ml to about 100 mg/ml; (ii) histidine at a concentration of about 20 mM±2 mM; (iii) sucrose at a concentration of about 10% w/v±1% w/v; and (iv) polysorbate 20 at a concentration of about 0.2% w/v±0.02% w/v.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) about 10 mM±1 mM histidine; (iii) about 5% w/v±0.5% w/v sucrose; and (iv) about 0.1%±0.01% w/v polysorbate 20, wherein the formulation has a pH of 6.0±0.1.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of from about 25 mg/ml to about 100 mg/ml; (ii) about 20 mM±2 mM histidine; (iii) about 10% w/v±1% w/v sucrose; and (iv) about 0.2%±0.02% w/v polysorbate 20, wherein the formulation has a pH of 6.0±0.1.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of about 50 mg/ml±5 mg/ml; (ii) about 10 mM±1 mM histidine; (iii) about 5% w/v±0.5% w/v sucrose; and (iv) about 0.1%±0.01% w/v polysorbate 20, wherein the formulation has a pH of from 6.0±0.1.

In some cases, the stable pharmaceutical formulation comprises, in an aqueous solution, (i) a human antibody that specifically binds to hCTLA-4 and comprises a HCVR comprising the amino acid sequence of SEQ ID NO: 1 and a LCVR comprising the amino acid sequence of SEQ ID NO: 2 at a concentration of about 100 mg/ml±10 mg/ml; (ii) about 20 mM±2 mM histidine; (iii) about 10% w/v±1% w/v sucrose; and (iv) about 0.2%±0.02% w/v polysorbate 20, wherein the formulation has a pH of from 6.0±0.1.

In some cases, the stable pharmaceutical formulation comprises, in an aqueous solution, (i) a human antibody that specifically binds to hCTLA-4 and comprises a HCVR comprising the amino acid sequence of SEQ ID NO: 1, a LCVR comprising the amino acid sequence of SEQ ID NO: 2 and a human IgG1 heavy chain constant region at a concentration of about 50 mg/ml±5 mg/ml; (ii) about 10 mM±1 mM histidine; (iii) about 5% w/v±0.5% w/v sucrose; and (iv) about 0.1%±0.01% w/v polysorbate 20, wherein the formulation has a pH of from 6.0±0.1.

In some cases, the stable pharmaceutical formulation comprises, in an aqueous solution, (i) a human antibody that specifically binds to hCTLA-4 and comprises a HCVR comprising the amino acid sequence of SEQ ID NO: 1, a LCVR comprising the amino acid sequence of SEQ ID NO: 2 and a human IgG1 heavy chain constant region at a concentration of about 100 mg/ml±10 mg/ml; (ii) about 20 mM±2 mM histidine; (iii) about 10% w/v±1% w/v sucrose; and (iv) about 0.02%±0.02% w/v polysorbate 20, wherein the formulation has a pH of from 6.0±0.1.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of about 50 mg/ml±5 mg/ml; (ii) about 10 mM±1 mM histidine; (iii) about 5% w/v±0.5% w/v sucrose; and (iv) about 0.1%±0.01% w/v polysorbate 20, wherein the formulation has a pH of from 6.0±0.1.

In some cases, the stable pharmaceutical formulation comprises, in an aqueous solution, (i) a human antibody that specifically binds to hCTLA-4 and comprises a heavy chain comprising the amino acid sequence of SEQ ID NO: 9 and a light chain comprising the amino acid sequence of SEQ ID NO: 10 at a concentration of about 100 mg/ml±10 mg/ml; (ii) about 20 mM±2 mM histidine; (iii) about 10% w/v±1% w/v sucrose; and (iv) about 0.02%±0.02% w/v polysorbate 20, wherein the formulation has a pH of from 6.0±0.1.

Additional non-limiting examples of pharmaceutical formulations encompassed by the present invention are set forth elsewhere herein, including the working Examples presented below.

### Stability of the Pharmaceutical Formulations

The pharmaceutical formulations of the present invention exhibit high levels of stability. The term “stable,” as used herein in reference to the pharmaceutical formulations, means that the antibodies within the pharmaceutical formulations retain an acceptable degree of structure and/or function and/or biological activity after storage for a defined amount of time. A formulation may be stable even though the antibody contained therein does not maintain 100% of its structure and/or function and/or biological activity after storage for a defined amount of time. Under certain circumstances, maintenance of about 90%, about 95%, about 96%, about 97%, about 98% or about 99% of an antibody's structure and/or function and/or biological activity after storage for a defined amount of time may be regarded as “stable.”

Stability can be measured by, inter alia, determining the percentage of native antibody remaining in the formulation after storage for a defined amount of time at a given temperature. The percentage of native antibody can be determined by, inter alia, size exclusion chromatography (e.g., size exclusion high performance liquid chromatography [SE-HPLC]). An “acceptable degree of stability,” as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 50° C., e.g., storage at about −80° C., about −30° C., about −20° C., about 0° C., about 4°−8° C., about 5° C., about 25° C., about 35° C., about 37° C., about 45° C., or about 50° C. For example, a pharmaceutical formulation may be deemed stable if after 3 months of storage at 5° C., greater than about 90%, 95%, 96% or 97% of native antibody is detected by SE-HPLC. A pharmaceutical formulation may also be deemed stable if after 6 months of storage at 5° C., greater than about 90%, 95%, 96% or 97% of native antibody is detected by SE-HPLC. A pharmaceutical formulation may also be deemed stable if after 9 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 24 months of storage at 5° C., greater than about 90%, 95%, 96%, 96.5%, or 97% of native antibody is detected by SE-HPLC. A pharmaceutical formulation may also be deemed stable if after 3 months of storage at 25° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 6 months of storage at 25° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 9 months of storage at 25° C., greater than about 90%, 95%, 96%, 96.5%, 97%, 97.5%, 98%, 98.5%, 99% or 99.5% of native antibody is detected by SE-HPLC. In certain embodiments, a “stable” pharmaceutical composition or pharmaceutical formulation of the present invention comprises at least 95%, at least 96%, or at least 97% native form of the antibody, as measured by size exclusion ultra-performance liquid chromatography (SE-UPLC) after six months of storage at 25° C. and 60% relative humidity.

Other methods may be used to assess the stability of the formulations of the present invention such as, e.g., differential scanning calorimetry (DSC) to determine thermal stability, controlled agitation to determine mechanical stability, and absorbance at about 350 nm or about 405 nm to determine solution turbidities. For example, a formulation of the present invention may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at t=0.

Measuring the binding affinity of the antibody to its target may also be used to assess stability. For example, a formulation of the present invention may be regarded as stable if, after storage at e.g., −80° C., −30° C., −20° C., 5° C., 25° C., 37° C., 45° C., etc. for a defined amount of time (e.g., 14 days to 9 months), the anti-CTLA-4 antibody contained within the formulation binds to hCTLA-4 with an affinity that is at least 80%, 85%, 90%, 95%, or more of the binding affinity of the antibody prior to said storage. Binding affinity may be determined by any method, such as e.g., ELISA or plasmon resonance. Biological activity may be determined by an CTLA-4 activity assay, such as by contacting a cell that expresses CTLA-4 with the formulation comprising the anti-CTLA-4 antibody. The binding of the antibody to such a cell may be measured directly, such as via FACS analysis. Alternatively, the downstream activity of the CTLA-4 system may be measured in the presence of the antibody, and compared to the activity of the CTLA-4 system in the absence of antibody.

Stability can be measured, inter alia, by determining the percentage of antibody that forms an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC] or size exclusion ultra-performance liquid chromatography [SE-UPLC]). An “acceptable degree of stability”, as that phrase is used herein, means that at most 6% of the antibody is in an aggregated form detected in the formulation after storage for a defined amount of time at a given temperature. In certain embodiments an acceptable degree of stability means that at most about 6%, 5.5%, 5%, 4.5%, 4%, 3.5%, 3%, 2.5%, 2%, 1.5%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 50° C., e.g., storage at about −80° C., about −30° C., about −20° C., about 0° C., about 4°−8° C., about 5° C., about 25° C., about 35° C., about 37° C. about 45° C., or about 50° C. For example, a pharmaceutical formulation may be deemed stable if after nine months of storage at 5° C., less than about 2%, 1.75%, 1.5%, 1.25%, 1%, 0.75%, 0.5%, 0.25%, or 0.1% of the antibody is detected in an aggregated form. A pharmaceutical formulation may also be deemed stable if after six months of storage at 25° C., less than about 2.5%, 2%, 1.75%, 1.5%, 1.25%, 1%, 0.75%, 0.5%, 0.25%, or 0.1% of the antibody is detected in an aggregated form. A pharmaceutical formulation may also be deemed stable if after 28 days of storage at 45° C., less than about 4%, 3.5%, 3%, 2.5%, 2%, 1.5%, 1%, 0.5%, or 0.1% of the antibody is detected in an aggregated form. A pharmaceutical formulation may also be deemed stable if after three months of storage at −20° C., −30° C., or −80° C. less than about 2%, 1.9%, 1.8%, 1.7%, 1.6%, 1.5%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” antibody can be determined by ion exchange chromatography (e.g., cation exchange high performance liquid chromatography [CEX-HPLC] or cation exchange ultra-performance liquid chromatography [CEX-UPLC]). An “acceptable degree of stability”, as that phrase is used herein, means that at most 60% of the antibody is in a more acidic form detected in the formulation after storage for a defined amount of time at a defined temperature. In certain embodiments an acceptable degree of stability means that at most about 55%, 50%, 45%, 40%, 35%, 30%, 29%, 28%, 27%, 26%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 50° C., e.g., storage at about −80° C., about −30° C., about −20° C., about 0° C., about 4°−8° C., about 5° C., about 25° C., about 45° C., or about 50° C. For example, a pharmaceutical formulation may be deemed stable if after three months of storage at −80° C., −30° C., or −20° C. less than about 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after nine months of storage at 5° C., less than about 50%, 45%, 40%, 35%, 30%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 25° C., less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 37° C., less than about 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after one month of storage at 50° C., less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

A “stable” formulation (including a liquid formulation, a lyophilized formulation, or a reconstituted formulation) is one in which the protein therein essentially retains its physical stability and/or chemical stability and/or biological activity upon storage for a period of time. A “stable” lyophilized formulation is a lyophilized formulation with no significant changes observed at a refrigerated temperature (e.g., 2-8° C.). In certain embodiments, a “stable” pharmaceutical composition or pharmaceutical formulation of the present invention comprises no more than 2%, no more than 1.9%, no more than 1.8%, no more than 1.7%, no more than 1.6%, or no more than 1.5% HMW species, as measured by size exclusion ultra-performance liquid chromatography (SE-UPLC) after, one, three, six, nine, twelve, 18, or 24 months of storage at 5° C. In certain embodiments, a “stable” pharmaceutical composition or pharmaceutical formulation of the present invention comprises no more than 2%, no more than 1.9%, no more than 1.8% HMW species, as measured by SE-UPLC after one month of storage at 45° C. In certain embodiments, a “stable” pharmaceutical composition or pharmaceutical formulation of the present invention comprises no more than 3% HMW species, as measured by SE-UPLC after one month of storage at 50° C. In certain embodiments, a “stable” pharmaceutical composition or pharmaceutical formulation of the present invention comprises no more than 2.5%, no more than 2.4%, no more than 2.3%, no more than 2.2%, no more than 2.1%, no more than 2%, no more than 1.9%, no more than 1.8%, no more than 1.7%, no more than 1.6%, no more than 1.5%, or no more than 1.4% HMW species, as measured by SE-UPLC after one, three or six months of storage at 25° C. and 60% relative humidity. In certain embodiments, a “stable” pharmaceutical composition or pharmaceutical formulation of the present invention comprises no more than 2%, no more than 1.9%, no more than 1.8%, no more than 1.7%, no more than 1.6%, or no more than 1.5% HMW species, as measured by SE-UPLC after 30 or 60 minutes of agitation at ambient temperature (e.g., 25° C.).

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 9 months means that the measurement of stability was taken at or about 9 months after the start of the study. Additional methods for assessing the stability of an antibody in formulation are demonstrated in the Examples presented below.

As illustrated in the Examples below, the present invention is based, in part, on the discovery that the combination of claimed excipients with an anti-CTLA-4 antibody produces a formulation that is stable and/or has an acceptable reconstitution time (e.g., less than 10 minutes, less than 5 minutes, less than 4 minutes, less than 3 minutes, etc.).

### Containers and Methods of Administration

The pharmaceutical formulations of the present invention may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present invention including, e.g., clear and opaque (e.g., amber) glass or plastic vials. Likewise, any type of syringe can be used to contain and/or administer the pharmaceutical formulations of the present invention. In some embodiments, the pharmaceutical formulation is contained in a glass vial. In some embodiments, the pharmaceutical formulation is contained in a prefilled syringe. In some embodiments, the pharmaceutical formulation is contained in a prefilled staked needle syringe.

The pharmaceutical formulations of the present invention may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present invention, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present invention, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present invention are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present invention are commercially available under the tradename “FluroTec®,” available from West Pharmaceutical Services, Inc. (Lionville, Pa.). According to certain embodiments of the present invention, the pharmaceutical formulations may be contained within a low tungsten syringe that comprises a fluorocarbon-coated plunger. In some embodiments, the container is a syringe, such as an Ompi EZ-Fill™ syringe or a BD Neopak™ syringe. In some cases, the syringe is a 1 mL long glass syringe with a 1 mL iWest piston, a 27G thin wall needle and an FM30 needle shield or a BD260 needle shield. In some cases, the syringe is a 2.25 mL glass syringe with a West NovaPure™ 1-3 mL piston, a 27G thin wall needle and an FM30 needle shield or a BD260 needle shield. In various embodiments, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 mL syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park, Ill.), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25 gauge needle. In some cases, subcutaneous injections may be administered using a 27 gauge needle. In some cases, subcutaneous injections may be administered using a 29 gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present invention is also contemplated herein. In some cases, the LVD or bolus injector may be configured to inject a medicament into a patient. For example, an LVD or bolus injector may be configured to deliver a “large” volume of medicament (typically about 2 ml to about 10 ml).

The pharmaceutical formulations of the present invention can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various embodiments, the unit dosage form is contained within a container as discussed herein. Actual dosage levels of the active ingredient (e.g., an anti-CTLA-4 antibody) in the formulations of the present invention may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various embodiments, the unit dosage form contains an amount of the active ingredient (e.g., an anti-CTLA-4 antibody) intended for a single use. In various embodiments, the amount of the active ingredient in the unit dosage form is from about 0.1 mg to about 5000 mg, from about 10 mg to about 1000 mg, and from about 10 mg to about 500 mg, from about 10 mg to about 400 mg, from about 10 mg to about 200 mg, from about 50 mg to about 150 mg, from about 250 mg to about 350 mg, from about 125 mg to about 175 mg, from about 275 mg to about 325 mg, or ranges or intervals thereof. Ranges intermediate to the above recited amounts, for example, from about 135 mg to about 165 mg or 285 mg to 315 mg, are also intended to be part of this invention. For example, ranges of values using a combination of any of the above recited values (or values contained within the above recited ranges) as upper and/or lower limits are intended to be included. In a particular embodiment, the formulation often is supplied as a liquid in unit dosage form. In some embodiments, a unit dosage form according to the present invention is suitable for subcutaneous administration to a patient. In some embodiments, a unit dosage form according to the present invention is suitable for intravenous administration to a patient.

The present invention also includes methods of preparing a unit dosage form. In an exemplary embodiment, a method for preparing a pharmaceutical unit dosage form includes combining the formulation of any of foregoing embodiments in a suitable container (e.g., those containers discussed herein).

### Therapeutic Uses of the Pharmaceutical Formulations

The pharmaceutical formulations of the present invention are useful, inter alia, for the treatment, prevention and/or amelioration of any disease or disorder associated with CTLA-4 activity. In particular, the pharmaceutical formulations of the invention are useful, inter alia, for the treatment, prevention and/or amelioration of any disease or disorder associated with or mediated by CTLA-4 expression, signaling, or activity, or treatable by blocking the interaction between CTLA-4 and a CTLA-4 ligand (e.g., ST2) or otherwise inhibiting CTLA-4 activity and/or signaling.

The therapeutic methods of the present invention comprise administering to a subject any formulation comprising an anti-hCTLA-4 antibody as disclosed herein. The subject to which the pharmaceutical formulation is administered can be, e.g., any human or non-human animal that is in need of such treatment, prevention and/or amelioration, or who would otherwise benefit from the inhibition or attenuation of CTLA-4 and/or CTLA-4-mediated activity. For example, the subject can be an individual that is diagnosed with, or who is deemed to be at risk of being afflicted by any of the aforementioned diseases or disorders. The present invention further includes the use of any of the pharmaceutical formulations disclosed herein in the manufacture of a medicament for the treatment, prevention and/or amelioration of any disease or disorder associated with CTLA-4 activity, including any of the above mentioned exemplary diseases, disorders and conditions.

In some embodiments, the present invention provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

### Exemplary Sequences

The sequences discussed herein and shown in the accompanying sequence listing correspond to mAb1, a fully human antibody with an IgG1 heavy chain constant region, which is used throughout the following Examples. The identities of the sequences are shown below. This antibody is also known as REGN4659.

Sequence Table (SEQ ID NOs)

| SEQ ID NO: | Description |
|---|---|
| 1 | HCVR |
| 2 | LCVR |
| 3 | HCDR1 |
| 4 | HCDR2 |
| 5 | HCDR3 |
| 6 | LCDR1 |
| 7 | LCDR2 |
| 8 | LCDR3 |
| 9 | Full Length Heavy Chain |
| 10 | Full Length Light Chain |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Effect of Buffer and pH on the Stability of an Anti-CTLA-4 Antibody

The effect of buffer and pH on the thermal stability of mAb1 liquid formulations was examined by incubating 25 mg/mL mAb1 at 45° C. for 28 days in a series of buffer systems at varying pH ranges. The following pH and buffer systems were studied: 10 mM acetate (pH 4.5 to 5.5), 10 mM histidine (pH 5.5 to 6.5), and 10 mM phosphate (pH 6.0 to 7.0). Based on results from SE-UPLC and CEX-UPLC analyses, maximum protein stability was observed when mAb1 was formulated between pH 5.5 and pH 6.5 in histidine buffer. These analyses also revealed that formation of low molecular weight (LMW) species, high molecular weight (HMW) species, and charge variants were the main degradation pathways. The 10 mM histidine buffer at pH 6.0 was selected as the formulation buffer for the drug product (DP) because it provided the best overall level of stabilization. The results are shown in Table 1.

TABLE 1
Effect of Buffer and pH on the Stability of 25 mg/mL mAb1 Incubated at 45° C. for 28 Days
25 mg/mL mAb1, 10 mM buffer
0.4 mL
2 mL Type 1 borosilicate glass vial with a FluroTec ®-coated 4432/50 butyl rubber stopper

| | | | % Total | | | | Change in Charged | | |
|---|---|---|---|---|---|---|---|---|---|
| | | Turbidity | Protein | Change in Purity | | | Variants | | |
| | | (Increase | Recovered | by SE-UPLC^{a} | | | by CEX-UPLC^{a} | | |
| pH/ | Color and | in OD at | by RP- | % | % | % | % | % | % |
| Buffer | Appearance | 405 nm) | UPLC | HMW | Native | LMW | Acidic | Main | Basic |
| pH 4.5, | Pass | 0.00 | 101 | 0.1 | −5.0 | 4.9 | 5.5 | −26.8 | 21.3 |
| Acetate | | | | | | | | | |
| pH 5.0, | Pass | 0.00 | 100 | 0.1 | −3.0 | 2.9 | 14.5 | −24.1 | 9.5 |
| Acetate | | | | | | | | | |
| pH 5.5, | Pass | 0.00 | 100 | 0.3 | −2.8 | 2.5 | 15.5 | −21.2 | 5.7 |
| Acetate | | | | | | | | | |
| pH 5.5, | Pass | 0.00 | 100 | −0.1 | −2.4 | 2.5 | 11.8 | −19.4 | 7.7 |
| Histidine | | | | | | | | | |
| pH 6.0, | Pass | 0.01 | 100 | 0.0 | −2.3 | 2.4 | 12.8 | −17.4 | 4.7 |
| Histidine | | | | | | | | | |
| pH 6.5, | Pass | 0.00 | 100 | 0.3 | −3.3 | 2.9 | 16.0 | −20.7 | 4.7 |
| Histidine | | | | | | | | | |
| pH 6.0, | Pass | 0.00 | 100 | 1.2 | −3.7 | 2.5 | 16.5 | −21.8 | 5.4 |
| Phosphate | | | | | | | | | |
| pH 6.5, | Pass | 0.00 | 100 | 2.0 | −4.8 | 2.7 | 18.9 | −24.4 | 5.5 |
| Phosphate | | | | | | | | | |
| pH 7.0, | Pass | 0.00 | 99 | 3.7 | −7.1 | 3.4 | 26.2 | −33.0 | 6.8 |
| Phosphate | | | | | | | | | |

^{a}Reported as a change in purity relative to the starting material. The starting material (no incubation) contains ≥95.6% native peak by SE-UPLC and ≥47.1% main peak by CEX-UPLC in all formulations.
CEX, cation exchange;
HMW, high molecular weight;
LMW, low molecular weight;
OD, optical density;
RP, reversed-phase;
SE, size-exclusion;
UPLC, ultra performance liquid chromatography

### Example 2: Effect of Thermal Stabilizers on the Stability of an Anti-CTLA-4 Antibody

Stabilizers such as sucrose may be added to antibody formulations to stabilize the protein to freezing and thawing stress and to increase the thermal stability of the protein in liquid and lyophilized formulations. The effect of sucrose on the freeze/thaw stability of mAb1 liquid formulations was examined by exposing 25 mg/mL mAb1, with and without 10% sucrose, to eight freeze/thaw cycles. A 1.0% increase in HMW species was observed by SE-UPLC for the formulation without sucrose, whereas no appreciable increase in HMW was observed by SE-UPLC for the formulation containing sucrose, as shown in Table 2. The effect of sucrose on the thermal stability of mAb1 liquid formulations was examined by incubating 25 mg/mL mAb1, formulated with and without 10% sucrose, at 45° C. for 28 days. No appreciable increase in HMW was observed by SE-UPLC irrespective of the presence of sucrose, as shown in Table 3.

TABLE 2
Effect of Sucrose on the Stability of 25 mg/mL mAb1 after Eight Freeze and Thaw Cycles
25 mg/mL mAb1, 10 mM histidine, pH 6.0
0.4 mL
2 mL Type 1 borosilicate glass vial with a FluroTec ®-coated 4432/50 butyl rubber stopper

| | | | | % Total | | | | Change in Charge | | |
|---|---|---|---|---|---|---|---|---|---|---|
| | | Turbidity | | Protein | Change in Purity | | | Variants | | |
| | | (Increase | | Recovered | by SE-UPLC^{a} | | | by CEX-UPLC^{a} | | |
| | Color and | in OD at | | by RP- | % | % | % | % | % | % |
| Formulation | Appearance | 405 nm) | pH | UPLC | HMW | Native | LMW | Acidic | Main | Basic |
| No sucrose | Pass | 0.01 | 6.2 | 98 | 1.0 | −1.0 | 0.0 | −0.6 | 0.2 | 0.4 |
| 10% (w/v) | Pass | 0.01 | 6.2 | 99 | 0.0 | 0.0 | 0.0 | −0.1 | 0.1 | −0.1 |
| sucrose | | | | | | | | | | |

^{a}Reported as a change in purity relative to the starting material. The starting material (no incubation) contains ≥97.1% native peak by SE-UPLC and ≥47.9% main peak by CEX-UPLC in all formulations.
CEX, cation exchange;
HMW, high molecular weight;
LMW, low molecular weight;
OD, optical density;
RP, reversed-phase;
SE, size-exclusion;
UPLC, ultra performance liquid chromatography

TABLE 3
Effect of Sucrose on the Stability of 25 mg/mL mAb1 Incubated at 45° C. for 28 Days
25 mg/mL mAb1, 10 mM histidine, pH 6.0
0.4 mL
2 mL Type 1 borosilicate glass vial with a FluorTec ®-coated 4432/50 butyl rubber stopper

| | | | | % Total | | | | Change in Charge | | |
|---|---|---|---|---|---|---|---|---|---|---|
| | | Turbidity | | Protein | Change in Purity | | | Variants | | |
| | | (Increase | | Recovered | by SE-UPLC^{a} | | | by CEX-UPLC^{a} | | |
| | Color and | in OD at | | by RP- | % | % | % | % | % | % |
| Formulation | Appearance | 405 nm) | pH | UPLC | HMW | Native | LMW | Acidic | Main | Basic |
| No sucrose | Pass | 0.01 | 6.2 | 101 | 0.2 | −2.9 | 2.7 | 13.1 | −17.8 | 4.6 |
| 10% (w/v) | Pass | 0.01 | 6.2 | 100 | 0.1 | −2.5 | 2.5 | 15.4 | −19.5 | 4.1 |
| sucrose | | | | | | | | | | |

^{a}Reported as a change in purity relative to the starting material. The starting material (no incubation) contains ≥97.1% native peak by SE-UPLC and ≥47.9% main peak by CEX-UPLC in all formulations
CEX, cation exchange;
HMW, high molecular weight;
LMW, low molecular weight;
OD, optical density;
RP, reversed-phase;
SE, size-exclusion;
UPLC, ultra performance liquid chromatography

5% sucrose was selected as the lyoprotectant in the formulated drug substance (FDS) because it was shown to provide sufficient mAb1 stability in the lyophilized DP (see Table 4, below). In the lead formulation, no differences were observed between the FDS (prior to lyophilization) and the reconstituted DP (post-lyophilization). Furthermore, DP reconstituted to 100 mg/mL mAb1 (containing 10% sucrose) yielded a nearly isotonic formulation.

TABLE 4
Effect of Sucrose Concentration on the Stability of Lyophilized mAb1 Drug Product
Incubated at 50° C. for 28 Days
50 mg/mL mAb1, 10 mM histidine, pH 6.0, 0.1% (w/v) polysorbate 20
0.4 mL
2 mL Type 1 borosilicate glass vial with a FluorTec ®-coated 4432/50 butyl rubber stopper

| | | | | % Total | | | | Change in Charge | | |
|---|---|---|---|---|---|---|---|---|---|---|
| | | Turbidity | | Protein | Change in Purity | | | Variants | | |
| | | (Increase | | Recovered | by SE-UPLC^{a} | | | by CEX-UPLC^{a} | | |
| Thermal | Color and | in OD at | | by RP- | % | % | % | % | % | % |
| Stabilizer | Appearance | 405 nm) | pH | UPLC | HMW | Native | LMW | Acidic | Main | Basic |
| No sucrose | Pass | 0.01 | 6.1 | 93 | 23.3 | −23.1 | −0.3 | −4.5 | −34.9 | 39.3 |
| 5% (w/v) | Pass | 0.00 | 6.1 | 100 | 1.3 | −1.0 | −0.4 | 0.2 | −3.8 | 3.6 |
| sucrose | | | | | | | | | | |

^{a}Reported as a change in purity relative to the starting material. The starting material (no incubation) contains ≥98.5% native peak by SE-UPLC and ≥54.6% main peak by CEX-UPLC in all four formulations.
CEX, cation exchange;
HMW, high molecular weight;
LMW, low molecular weight;
OD, optical density;
RP, reversed-phase;
SE, size-exclusion;
UPLC, ultra performance liquid chromatography

### Example 3: Effect of Surfactants on the Stability of an Anti-CTLA-4 Antibody

Stabilizers such as surfactants may be added to antibody formulations to protect the protein from surface-induced degradation. The effect of surfactants on the thermal stability of 25 mg/mL mAb1 was examined in liquid formulations. The following surfactants were evaluated: 0.1% polysorbate 20 and 0.1% polysorbate 80. The results of the thermal stability study are summarized in Table 5. When incubated at 45° C., the addition of polysorbate 20 or polysorbate 80 had a negative impact on the thermal stability of mAb1 relative to a control formulation lacking any surfactant. An increase in turbidity, LMW species, HMW species, and charge variants were observed. The formulation containing 0.1% polysorbate 20 formed the least amount of aggregates and charge variant species compared to the other formulations containing surfactant.

The effect of polysorbate 20 on the agitation stability of mAb1 was examined by vortexing 50 mg/mL mAb1, with and without polysorbate 20, for 120 minutes. A 2.5% increase in HMW species was observed by SE-UPLC for the formulation without polysorbate 20, whereas no appreciable increase in HMW was observed by SE-UPLC for the formulation containing polysorbate 20, as shown in Table 6.

TABLE 5
Effect of Organic Co-solvents and Surfactants on the Stability of 25 mg/mL mAb1
Incubated at 45° C. for 28 Days
25 mg/mL mAb1, 10 mM histidine, 10% (w/v) sucrose, pH 6.0
0.4 mL
2 mL Type 1 borosilicate glass vial with a FluroTec ®-coated 4432/50 butyl rubber stopper

| | | | | % Total | | | | Change in Charge | | |
|---|---|---|---|---|---|---|---|---|---|---|
| | | Turbidity | | Protein | Change in Purity | | | Variants | | |
| | | (Increase | | Recovered | by SE-UPLC^{a} | | | by CEX-UPLC^{a} | | |
| | Color and | in OD at | | by RP- | % | % | % | % | % | % |
| Surfactant | Appearance | 405 nm) | pH | UPLC | HMW | Native | LMW | Acidic | Main | Basic |
| No | Pass | 0.01 | 6.2 | 100 | 0.1 | −2.5 | 2.5 | 15.4 | −19.5 | 4.1 |
| surfactant | | | | | | | | | | |
| 0.1% (w/v) | Pass | 0.05 | 5.9 | 97 | 2.5 | −5.5 | 3.1 | 24.7 | −25.6 | 0.9 |
| polysorbate | | | | | | | | | | |
| 20 | | | | | | | | | | |
| 0.1% (w/v) | Pass | 0.05 | 6.0 | 99 | 3.4 | −6.5 | 3.1 | 31.3 | −31.2 | −0.1 |
| polysorbate | | | | | | | | | | |
| 80 | | | | | | | | | | |

^{a}Reported as a change in purity relative to the starting material. The starting material (no incubation) contains ≥97.1% native peak by SE-UPLC and ≥47.9% main peak by CEX-UPLC in all formulations.
CEX, cation exchange;
HMW, high molecular weight;
LMW, low molecular weight;
OD, optical density;
RP, reversed-phase;
SE, size-exclusion;
UPLC, ultra performance liquid chromatography

TABLE 6
Effect of Polysorbate 20 on the Stability of 50 mg/mL mAb1 Following Agitation (120 Minutes of Vortexing)
50 mg/mL mAb1, 10 mM histidine, pH 6.0, 5% (w/v) sucrose
0.4 mL
2 mL Type 1 borosilicate glass vial with a FluorTec ®-coated 4432/50 butyl rubber stopper

| | | | | % Total | | | | Change in Charge | | |
|---|---|---|---|---|---|---|---|---|---|---|
| | | Turbidity | | Protein | Change in Purity | | | Variants | | |
| | | (Increase | | Recovered | by SE-UPLC^{a} | | | by CEX-UPLC^{a} | | |
| | Color and | in OD at | | by RP- | % | % | % | % | % | % |
| Surfactant | Appearance | 405 nm) | pH | UPLC | HMW | Native | LMW | Acidic | Main | Basic |
| No | Pass | 0.00 | 6.1 | 100 | 2.5 | −2.3 | 0.0 | −0.7 | 0.6 | 0.2 |
| surfactant | | | | | | | | | | |
| 0.1% (w/v) | Pass | 0.00 | 6.1 | 100 | 0.1 | −0.1 | 0.1 | −0.2 | 0.2 | 0.0 |
| polysorbate | | | | | | | | | | |
| 20 | | | | | | | | | | |

^{a}Reported as a change in purity relative to the starting material. The starting material (no incubation) contains ≥96.7% native peak by SE-UPLC and ≥47.8% main peak by CEX-UPLC in all formulations.
CEX, cation exchange;
HMW, high molecular weight;
LMW, low molecular weight;
OD, optical density;
RP, reversed-phase;
SE, size-exclusion;
UPLC, ultra performance liquid chromatography

### Example 4: Stability of Liquid, Lyophilized, and Reconstituted Formulations of Anti-CTLA-4 Antibody Drug Product

Studies to evaluate the storage, accelerated stability, and stress stability (agitation) of liquid, lyophilized, and reconstituted formulations of mAb1 drug product were undertaken. The lyophilized DP used for the storage and stability studies was manufactured by lyophilizing 5.3 mL of FDS in 20 mL Type 1 glass vials according to the lyophilization cycle discussed in Example 5. The reconstituted formulations were prepared by reconstituting lyophilized DP (5.3 mL FDS in 20 mL type 1 glass vials) to 50 mg/mL mAb1 with 4.9 mL water for injection (WFI) and to 100 mg/mL mAb1 with 2.3 mL WFI (for intravenous or subcutaneous administration).

The stability of mAb1 DP formulations was assessed using the following assays:
For lyophilized DP samples
   Cake appearance by visual inspection of lyophilized DP
   Moisture content of lyophilized DP by Vapor Pro® moisture analyzer
   Reconstitution time of lyophilized DP
For liquid samples, including reconstituted DP samples
   Color and appearance by visual inspection
   pH
   Turbidity measured by increase in optical density (OD) at 405 nm
   Subvisible particulate analysis by Micro-Flow Imaging (MFI)
   Protein recovered by reversed-phase ultra performance liquid chromatography (RP-UPLC)
   Purity by the following assays:
Reduced and non-reduced microchip capillary electrophoresis (MCE);
Size-exclusion ultra performance liquid chromatography (SE-UPLC)
   Charge variant analysis by the following assays:
      Cation exchange (CEX) UPLC
      Imaged capillary isoelectric focusing (iCIEF)
   % Relative potency:
      The relative potency of each sample is determined using a bioassay and is defined as: (IC₅₀ Reference Sample/Ca) Sample)×100%. The measured potency of storage stability samples must be within 50 to 150% of the measured potency of the reference standard.

Results from the storage and stability studies are shown in Tables 7 to 13, below.

TABLE 7
Research Stability of 50 mg/mL mAb1 Liquid Drug Product
Stored at 5° C. - 1.5 mL Fill in 2 mL Glass Vial

| Pre-Lyophilized Formulation | | 50 mg/mL mAb1, 10 mM histidine, pH 6.0, 5% (w/v) sucrose, 0.1% (w/v) | | | | | | | | |
|---|---|---|---|---|---|---|---|---|---|---|
| | | polysorbate 20 | | | | | | | | |
| Fill Volume | | 1.5 mL | | | | | | | | |
| Container/Closure | | 2 mL Type 1 borosilicate glass vials with 13 mm FluroTec ® coated West | | | | | | | | |
| | | S2-F452 4432/50 GRY B2-40 stoppers | | | | | | | | |
| | | Length of Storage at 5° C. (months) | | | | | | | | |
| Assay | | 0 | 1 | 3 | 6 | 9 | 12 | 18 | 24 | 36 |
| Color and Appearance | | Pass | Pass | Pass | Pass | Pass | Pass | Pass | Pass | Pass |
| Turbidity (Increase in | | 0.00 | 0.00 | 0.00 | 0.00 | 0.00 | 0.00 | 0.00 | 0.00 | 0.00 |
| OD at 405 nm) | | | | | | | | | | |
| pH | | 6.1 | 6.0 | 6.1 | 6.1 | 6.1 | 6.1 | 6.1 | 6.1 | 6.0 |
| Particulate | 2-10 μm | 626 | NR | NR | 3676 | NR | ND | NR | 10644 | 4037 |
| Analysis | ≥10 μm | 33 | NR | NR | 129 | NR | ND | NR | 302 | 104 |
| by MFI | ≥25 μm | 2 | NR | NR | 17 | NR | ND | NR | 31 | 10 |
| % Total Protein Recovered | | 100 | 99 | 96 | 97 | 98 | 97 | 97 | 99 | 98 |
| by RP-UPLC | | | | | | | | | | |
| Purity by | % HMW | 1.4 | 1.3 | 1.4 | 1.5 | 1.5 | 1.6 | 1.6 | 1.6 | 1.5 |
| SE-UPLC | % Native | 97.8 | 97.8 | 97.6 | 97.4 | 97.4 | 97.1 | 97.0 | 97.1 | 97.2 |
| | % LMW | 0.8 | 0.8 | 1.0 | 1.1 | 1.1 | 1.3 | 1.4 | 1.3 | 1.3 |
| Charge | % Acidic | 43.8 | 43.8 | 43.0 | 40.6 | 40.5 | 40.7 | 40.5 | 43.1 | 43.9 |
| Variant | % Main | 50.3 | 50.4 | 50.4 | 54.6 | 54.6 | 54.4 | 54.6 | 50.8 | 51.6 |
| Analysis by | % Basic | 5.9 | 5.8 | 6.6 | 4.8 | 4.9 | 4.9 | 4.8 | 6.2 | 4.5 |
| CEX-UPLC | | | | | | | | | | |

CEX, cation exchange;
DS, drug substance,
HMW, high molecular weight;
iCIEF, image capillary isoelectric focusing;
LMW, low molecular weight;
MCE, microchip capillary electrophoresis;
MFI, Micro-Flow Imaging;
NR, not required per protocol;
ND, not determined;
OD, optical density;
RP, reversed-phase;
SE, size-exclusion;
UPLC, ultra performance liquid chromatography

TABLE 8
Research Stability of 50 mg/mL mAb1 Liquid Drug Product - 1.5
mL Fill in 2 mL Glass Vial - Effect of Accelerated Conditions

| Formulation | | 50 mg/mL mAb1, 10 mM histidine, pH 6.0, 5% (w/v) sucrose, 0.1% (w/v) | | | | | | |
|---|---|---|---|---|---|---|---|---|
| | | polysorbate 20 | | | | | | |
| Fill Volume | | 1.5 mL | | | | | | |
| Container/Closure | | 2 mL Type 1 borosilicate glass vials with 13 mm FluroTec ® coated West | | | | | | |
| | | S2-F452 4432/50 GRY B2-40 stoppers | | | | | | |
| | | | 25° C./60% RH Storage | | | | | |
| | | | (months) | | | 45° C. Storage (months) | | |
| Assay | | 0 | 1 | 3 | 6 | 0.5 | 1 | 3 |
| Color and Appearance | | Pass | Pass | Pass | Pass | Pass | Pass | Pass |
| Turbidity (Increase in | | 0.00 | 0.01 | 0.01 | 0.01 | 0.02 | 0.02 | 0.04 |
| OD at 405 nm) | | | | | | | | |
| pH | | 6.1 | 6.0 | 6.1 | 6.1 | 6.1 | 6.0 | 6.1 |
| Particulate | 2-10 μm | 625.8 | NR | NR | 1765 | NR | NR | 1556 |
| analysis | >10 μm | 33.4 | NR | NR | 77 | NR | NR | 65 |
| by MFI | >25 μm | 2.1 | NR | NR | 6 | NR | NR | 13 |
| (particles/mL) | | | | | | | | |
| % Protein Recovered | | 100 | 98 | 97 | 98 | 100 | 98 | 98 |
| by RP-UPLC | | | | | | | | |
| Purity by | % HMW | 1.4 | 1.4 | 1.5 | 1.7 | 1.5 | 1.8 | 3.3 |
| SE-UPLC | % Native | 97.8 | 97.6 | 97.0 | 96.1 | 96.6 | 95.0 | 89.5 |
| | % LMW | 0.8 | 1.0 | 1.5 | 2.2 | 1.9 | 2.4 | 5.1 |
| Charge | % Acidic | 43.8 | 43.8 | 45.0 | 46.4 | 52.1 | 61.7 | 79.0 |
| Variant | % Main | 50.3 | 50.4 | 48.6 | 47.8 | 38.3 | 27.7 | 10.5 |
| Analysis by | % Basic | 5.9 | 5.8 | 6.4 | 5.8 | 9.7 | 10.6 | 10.5 |
| CEX-UPLC | | | | | | | | |

CEX, cation exchange;
DS, drug substance, HMW, high molecular weight;
iCIEF, image capillary isoelectric focusing;
LMW, low molecular weight;
MCE, microchip capillary electrophoresis;
MFI, Micro-Flow Imaging;
NR, not required per protocol;
OD, optical density;
RP, reversed-phase;
SE, size-exclusion;
UPLC, ultra performance liquid chromatography

TABLE 9
Research Stability of 50 mg/mL mAb1 Liquid Drug Product - 1.5
mL Fill in 2 mL Glass Vial - Effect of Stress Conditions

| Formulation | | 50 mg/mL mAb1, 10 mM histidine, pH 6.0, 5% (w/v) sucrose, 0.1% (w/v) | | |
|---|---|---|---|---|
| | | polysorbate 20 | | |
| Fill Volume | | 1.5 mL | | |
| Container/Closure | | 2 mL Type 1 borosilicate glass vials with 13 mm FluroTec ® coated West | | |
| | | S2-F452 4432/50 GRY B2-40 stoppers | | |
| | | | Agitation (minutes) | |
| Assay | | 0 | 60 | 120 |
| Color and Appearance | | Pass | Pass | Pass |
| Turbidity (Increase | | 0.00 | 0.00 | 0.00 |
| in OD at 405 nm) | | | | |
| pH | | 6.1 | 6.1 | 6.1 |
| Particulate | 2-10 μm | 626 | NR | 536 |
| analysis by MFI | ≥10 μm | 33 | NR | 38 |
| (particles/mL) | ≥25 μm | 2 | NR | 8 |
| % Protein Recovered | | 100 | 99 | 100 |
| by RP-UPLC | | | | |
| Purity by | % HMW | 1.4 | 1.4 | 1.4 |
| SE-UPLC | % Native | 97.8 | 97.8 | 97.8 |
| | % LMW | 0.8 | 0.8 | 0.8 |
| Charge Variant | % Acidic | 43.8 | 43.8 | 43.7 |
| Analysis by | % Main | 50.3 | 50.3 | 50.4 |
| CEX-UPLC | % Basic | 5.9 | 5.8 | 5.9 |

CEX, cation exchange;
DS, drug substance,
HMW, high molecular weight;
iCIEF, image capillary isoelectric focusing;
LMW, low molecular weight;
MCE, microchip capillary electrophoresis;
MFI, Micro-Flow Imaging;
NR, not required per protocol;
OD, optical density;
RP, reversed-phase;
SE, size-exclusion;
UPLC, ultra performance liquid chromatography

TABLE 10
Research Stability of mAb1 Lyophilized Drug Product Stored at 5° C.

| Pre-Lyophilized Formulation | 50 mg/mL mAb1, 10 mM histidine, pH 6.0, 5% (w/v) sucrose, 0.1% (w/v) | | | | | | | | |
|---|---|---|---|---|---|---|---|---|---|
| | polysorbate 20 | | | | | | | | |
| Fill Volume | 5.3 mL | | | | | | | | |
| Container/Closure | 20 mL Type 1 borosilicate glass vials with a 20 mm FluroTec ®-coated | | | | | | | | |
| | closure, single vent lyo, 4432/50 stoppers | | | | | | | | |
| | Length of Storage at 5° C. (months) | | | | | | | | |
| Assay | 0 | 1 | 3 | 6 | 9 | 12 | 18 | 24 | 36 |

Analysis of Lyophilized Drug Product

| | | | | | | | | | |
|---|---|---|---|---|---|---|---|---|---|
| Cake Appearance | Pass | Pass | Pass | Pass | Pass | Pass | Pass | Pass | Pass |
| % Moisture | 0.0 | NR | NR | 0.3 | NR | 0.3 | NR | 0.4 | 0.3 |
| Reconstitution Time (minutes) | <3 | <4 | <4 | <3 | <3 | <4 | <3 | <3 | <4 |

Analysis of Reconstituted Drug Product^{a}

| Color and Appearance | | Pass | Pass | Pass | Pass | Pass | Pass | Pass | Pass | Pass |
|---|---|---|---|---|---|---|---|---|---|---|
| Turbidity (Increase in | | — | 0.00 | 0.00 | 0.01 | 0.01 | 0.00 | 0.01 | 0.00 | 0.00 |
| OD at 405 nm) | | | | | | | | | | |
| pH | | 6.1 | 6.0 | 6.0 | 6.0 | 6.0 | 6.1 | 6.1 | 6.1 | 6.1 |
| Particulate | 2-10 μm | 538 | NR | NR | 671 | NR | 1370 | NR | 2705 | 1384 |
| Analysis | ≥10 μm | 31 | NR | NR | 38 | NR | 8 | NR | 15 | 19 |
| by MFI | ≥25 μm | 0 | NR | NR | 0 | NR | 2 | NR | 2 | 2 |
| % Total Protein Recovered | | 100 | 101 | 101 | 103 | 99 | 100 | 98 | 96 | 100 |
| by RP-UPLC | | | | | | | | | | |
| Purity by MCE | Non-reduced; % | 99.0 | NR | NR | 99.0 | NR | ND | NR | ND | ND |
| | main peak | | | | | | | | | |
| | Reduced; % | 100 | NR | NR | 100 | NR | ND | NR | ND | ND |
| | heavy + | | | | | | | | | |
| | light chain | | | | | | | | | |
| Purity by | % HMW | 1.8 | 1.6 | 1.7 | 1.8 | 1.8 | 1.8 | 1.8 | 1.9 | 2.0 |
| SE-UPLC | % Native | 97.2 | 97.4 | 97.5 | 97.3 | 97.3 | 97.4 | 97.3 | 97.2 | 97.1 |
| | % LMW | 1.0 | 1.0 | 0.8 | 0.9 | 0.9 | 0.8 | 0.9 | 1.0 | 1.0 |
| Charge | % Acidic | 45.9 | 45.4 | 45.6 | 45.5 | 46.4 | 46.6 | 46.5 | 47.2 | 46.7 |
| Variant | % Main | 47.7 | 47.9 | 48.0 | 47.8 | 48.2 | 48.3 | 46.6 | 46.5 | 46.6 |
| Analysis by | % Basic | 6.4 | 6.8 | 6.5 | 6.6 | 5.3 | 5.2 | 6.9 | 6.3 | 6.7 |
| CEX-UPLC | | | | | | | | | | |
| Charge | % Acidic | 55.3 | NR | NR | 54.6 | NR | 55.8 | NR | 54.9 | 56.0 |
| Variant | % Main | 41.4 | NR | NR | 41.4 | NR | 39.0 | NR | 38.2 | 38.6 |
| Analysis by | % Basic | 3.3 | NR | NR | 4.0 | NR | 5.2 | NR | 6.9 | 5.4 |
| iCIEF | | | | | | | | | | |

| | | | | | | | | | |
|---|---|---|---|---|---|---|---|---|---|
| % Relative Potency | 137 | NR | NR | 123 | NR | ND | NR | ND | ND |

^{a}Samples were reconstituted with sterile WFI to 100 mg/mL mAb1.
CEX, cation exchange;
DS, drug substance, HMW, high molecular weight;
iCIEF, image capillary isoelectric focusing;
LMW, low molecular weight;
MCE, microchip capillary electrophoresis;
MFI, Micro-Flow Imaging;
NR, not required per protocol;
ND, not determined;
OD, optical density;
RP, reversed-phase;
SE, size-exclusion;
UPLC, ultra performance liquid chromatography

TABLE 11
Research Stability of mAb1 Lyophilized Drug Product Stored under Accelerated Conditions

| Pre-Lyophilized Formulation | 50 mg/mL mAb1, 10 mM histidine, pH 6.0, 5% (w/v) sucrose, 0.1% (w/v) | | | | |
|---|---|---|---|---|---|
| | polysorbate 20 | | | | |
| Fill Volume | 5.3 mL | | | | |
| Container/Closure | 20 mL Type 1 borosilicate glass vial with West V10-F597W 4432/50 GRY | | | | |
| | B2-TR stopper | | | | |
| | | 25° C./60% RH Storage | | | |
| | No Storage | (months) | | 50° C. Storage (months) | |
| Assay | t = 0 | 3 | 6 | 1 | 3 |

Analysis of Lyophilized Drug Product

| | | | | | |
|---|---|---|---|---|---|
| Cake Appearance | Pass | Pass | Pass | Pass | Pass |
| % Moisture | 0.0 | NR | 0.3 | NR | NR |
| Reconstitution Time (minutes) | <3 | <4 | <3 | <4 | <5 |

Analysis of Reconstituted Drug Product^{a}

| Color and Appearance | | Pass | Pass | Pass | Pass | Pass |
|---|---|---|---|---|---|---|
| Turbidity (Increase in | | — | 0.01 | 0.01 | 0.00 | 0.03 |
| OD at 405 nm) | | | | | | |
| pH | | 6.1 | 6.0 | 6.0 | 6.0 | 6.0 |
| Particulate | 2-10 μm | 538 | NR | 726 | NR | NR |
| Analysis | ≥10 μm | 31 | NR | 17 | NR | NR |
| by MFI | ≥25 μm | 0 | NR | 2 | NR | NR |
| % Total Protein Recovered | | 100 | 103 | 103 | 91 | 105 |
| by RP-UPLC | | | | | | |
| Purity by MCE | Non-reduced; % | 99.0 | NR | 99.0 | NR | 99.0 |
| | main peak | | | | | |
| | Reduced; % | 100 | NR | 100 | NR | 100 |
| | heavy + | | | | | |
| | light chain | | | | | |
| Purity by | % HMW | 1.8 | 2.0 | 2.2 | 3.0 | 4.3 |
| SE-UPLC | % Native | 97.2 | 97.2 | 96.9 | 96.0 | 94.8 |
| | % LMW | 1.0 | 0.8 | 0.9 | 0.9 | 0.9 |
| Charge | % Acidic | 45.9 | 45.7 | 45.2 | 45.8 | 46.4 |
| Variant | % Main | 47.7 | 47.6 | 46.6 | 44.1 | 40.6 |
| Analysis by | % Basic | 6.4 | 6.7 | 8.2 | 10.1 | 13.0 |
| CEX-UPLC | | | | | | |
| Charge | % Acidic | 55.3 | NR | 54.3 | NR | 53.8 |
| Variant | % Main | 41.4 | NR | 41.2 | NR | 36.8 |
| Analysis by | % Basic | 3.3 | NR | 4.6 | NR | 9.3 |
| iCIEF | | | | | | |

| | | | | | |
|---|---|---|---|---|---|
| % Relative Potency | 137 | NR | 112 | NR | 107 |

^{a}Samples were reconstituted with sterile WFI to 100 mg/mL mAb1.
CEX, cation exchange;
DS, drug substance, HMW, high molecular weight;
iCIEF, image capillary isoelectric focusing;
LMW, low molecular weight;
MCE, microchip capillary electrophoresis;
MFI, Micro-Flow Imaging;
NR, not required per protocol;
OD, optical density;
RP, reversed-phase;
SE, size-exclusion;
UPLC, ultra performance liquid chromatography

TABLE 12
Research Stability of mAb1 Reconstituted Drug Product

| Formulation | | 50 mg/mL mAb1, 10 mM Histidine, pH 6.0, 5% (w/v) sucrose, 0.1% (w/v) | | | | |
|---|---|---|---|---|---|---|
| | | polysorbate 20 | | | | |
| Fill Volume | | 5.3 mL | | | | |
| Container/Closure | | 20 mL Type 1 borosilicate glass vial with a 20 mm FluroTec ®-coated | | | | |
| | | closure, single vent lyophilization, 4432/50 stoppers | | | | |
| | | | Agitation (minutes) | | | |
| | | No Stress | at ~25° C. (ambient) | | 25° C. Storage (hours) | |
| Assay | | t=0 | 30 | 60 | 8 | 24 |
| Color and Appearance | | Pass | Pass | Pass | Pass | Pass |
| Turbidity (Increase in | | — | 0.00 | 0.00 | 0.00 | 0.00 |
| OD at 405 nm) | | | | | | |
| pH | | 6.1 | 6.1 | 6.1 | 6.1 | 6.1 |
| Particulate | 2-10 μm | 203 | NR | 858 | NR | 876 |
| Analysis | ≥10 μm | 0 | NR | 25 | NR | 88 |
| by MFI | ≥25 μm | 0 | NR | 2 | NR | 6 |
| % Total Protein Recovered | | 100 | 101 | 101 | 100 | 101 |
| by RP-UPLC | | | | | | |
| Purity by MCE | Non-reduced; % | 99.1 | NR | 99.2 | NR | 99.2 |
| | main peak | | | | | |
| | Reduced; % | 100 | NR | 100 | NR | 100 |
| | heavy + | | | | | |
| | light chain | | | | | |
| Purity by | % HMW | 1.6 | 1.6 | 1.6 | 1.5 | 1.5 |
| SE-UPLC | % Native | 96.6 | 96.6 | 96.6 | 96.6 | 96.7 |
| | % LMW | 1.8 | 1.8 | 1.8 | 1.9 | 1.8 |
| Charge | % Acidic | 46.2 | 46.0 | 46.2 | 46.0 | 45.9 |
| Variant | % Main | 47.6 | 47.7 | 47.6 | 47.8 | 47.8 |
| Analysis by | % Basic | 6.2 | 6.2 | 6.2 | 6.2 | 6.3 |
| CEX-UPLC | | | | | | |
| Charge | % Acidic | 55.1 | NR | 54.7 | NR | 54.1 |
| Variant | % Main | 41.7 | NR | 42.0 | NR | 42.5 |
| Analysis by | % Basic | 3.2 | NR | 3.3 | NR | 3.5 |
| iCIEF | | | | | | |
| % Relative Potency by | 136 | NR | 114 | NR | 116 | |

CEX, cation exchange;
DS, drug substance, HMW, high molecular weight;
iCIEF, image capillary isoelectric focusing;
LMW, low molecular weight;
MCE, microchip capillary electrophoresis;
MFI, Micro Flow Imaging;
NR, not required per protocol;
OD, optical density;
RP, reversed-phase;
SE, size-exclusion;
UPLC, ultra performance liquid chromatography

TABLE 13
Research Stability of mAb1 Reconstituted Drug Product

| Formulation | | 100 mg/mL mAb1, 20 mM Histidine, pH 6.0, 10% (w/v) sucrose, 0.2% | | | | |
|---|---|---|---|---|---|---|
| | | (w/v) polysorbate 20 | | | | |
| Fill Volume | | 2.7 mL | | | | |
| Container/Closure | | 20 mL Type 1 borosilicate glass vial with a 20 mm FluroTec ®-coated | | | | |
| | | closure, single vent lyophilization, 4432/50 stoppers | | | | |
| | | | Agitation (minutes) | | | |
| | | No Stress | at ~25° C. (ambient) | | 25° C. Storage (hours) | |
| Assay | | t = 0 | 30 | 60 | 8 | 24 |
| Color and Appearance | | Pass | Pass | Pass | Pass | Pass |
| Turbidity (Increase in | | — | 0.00 | 0.00 | 0.01 | 0.01 |
| OD at 405 nm) | | | | | | |
| pH | | 6.1 | 6.1 | 6.1 | 6.1 | 6.1 |
| Particulate | 2-10 μm | 538 | NR | 953 | NR | 930 |
| Analysis | ≥10 μm | 31 | NR | 31 | NR | 69 |
| by MFI | ≥25 μm | 0 | NR | 10 | NR | 4 |
| % Total Protein Recovered | | 100 | 97 | 97 | 98 | 99 |
| by RP-UPLC | | | | | | |
| Purity by MCE | Non-reduced; % | 99.0 | NR | 98.9 | NR | 99.0 |
| | main peak | | | | | |
| | Reduced; % | 100 | NR | 100 | NR | 100 |
| | heavy + | | | | | |
| | light chain | | | | | |
| Purity by | % HMW | 1.5 | 1.9 | 1.9 | 1.8 | 1.8 |
| SE-UPLC | % Native | 97.6 | 96.3 | 96.3 | 96.4 | 96.4 |
| | % LMW | 0.9 | 1.9 | 1.9 | 1.8 | 1.8 |
| Charge | % Acidic | 45.9 | 45.0 | 45.1 | 44.8 | 44.9 |
| Variant | % Main | 47.7 | 49.2 | 49.0 | 49.4 | 49.2 |
| Analysis by | % Basic | 6.4 | 5.8 | 5.9 | 5.8 | 5.9 |
| CEX-UPLC | | | | | | |
| Charge | % Acidic | 55.3 | NR | 54.6 | NR | 54.5 |
| Variant | % Main | 41.4 | NR | 41.5 | NR | 42.2 |
| Analysis by | % Basic | 3.3 | NR | 3.9 | NR | 3.4 |
| iCIEF | | | | | | |
| % Relative Potency | 137 | NR | 115 | NR | 99 | |

CEX, cation exchange;
DS, drug substance, HMW, high molecular weight;
iCIEF, image capillary isoelectric focusing;
LMW, low molecular weight;
MCE, microchip capillary electrophoresis;
MFI, Micro-Flow Imaging;
NR, not required per protocol;
OD, optical density;
RP, reversed-phase;
SE, size-exclusion;
UPLC, ultra performance liquid chromatography

The results from the DP storage and stability studies indicate that mAb1 is stable when formulated at concentrations from 25 mg/ml to 100 mg/ml with from 10 mM to 20 mM histidine (pH 6), from 5% w/v to 10% w/v sucrose, and from 0.1% w/v to 0.2% w/v polysorbate 20. The mAb1 formulations can withstand exposures to room temperature without compromising physical or chemical stability. The mAb1 protein is also stable when reconstituted to concentrations between 50 and 100 mg/mL in the exemplified formulations. Exposure of the reconstituted mAb1 DP to 25° C. for up to 24 hours will not compromise the integrity of the protein, nor will agitation of the reconstituted DP. The mAb1 formulations maintained potency, as determined by bioassay analysis, even after incubation under the accelerated conditions.

### Example 5: Lyophilization Cycle Development

The lyophilization process that was developed for clinical production consists of: freezing, primary drying, and secondary drying. The lyophilization process was developed using an FTS LyoStar™ Ill lyophilizer based on a partial cake collapse temperature (T_{c}) of −15.7° C., determined for the frozen formulated drug substance (FDS) using a freeze-dry microscope. During primary drying, the product temperature must not exceed the partial cake T_{c} to maintain cake integrity during the lyophilization cycle. The secondary drying process was developed to ensure the DP has low residual moisture content.

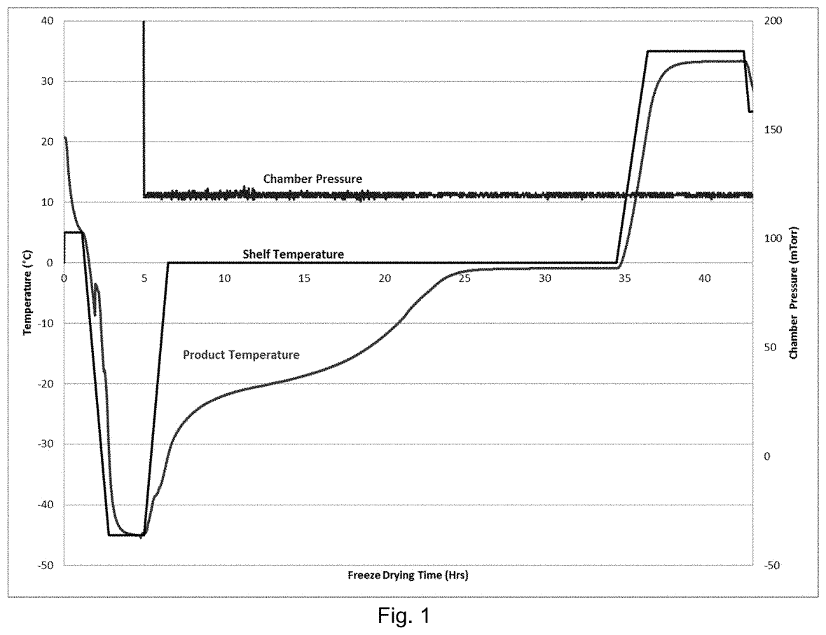

The lyophilization cycle takes approximately 43 hours to produce freeze-dried mAb1 DP in 20 mL Type 1 glass vials that were filled with 5.3 mL of 50 mg/mL mAb1 FDS. The lyophilization cycle (FIG. 1) contains the steps shown in Table 14, below.

TABLE 14
Lyophilization Cycle for mAb1 Drug Product

| | | Shelf | Holding | |
|---|---|---|---|---|
| | Shelf Temperature Ramp | Temperature | Time | |
| Lyophilization Step | Rate (Ramp Duration) | (° C.) | (hours) | Chamber Pressure |
| Loading | NA | 5-25 | NA | Ambient Pressure |
| Freezing | 0.5° C./minute | −45 | 2 | Ambient Pressure |
| | (100-140 minutes) | | | |
| Primary Drying | 0.5° C./minute (90 minutes) | 0 | 28 | 120 mTorr |
| Secondary Drying | 0.3° C./minute (117 minutes) | 35 | 6 | 120 mTorr |
| Temperature Ramp for | 0.5° C./minute (20 minutes) | 25 | 1 | 120 mTorr |
| Stoppering | | | | |
| Back Fill with Gas | NA | 25 | NA | 80% of Atmospheric |
| Nitrogen | | | | Pressure (608,000 mTorr) |
| Stoppering | NA | 25 | NA | 80% of Atmospheric |
| | | | | Pressure (608,000 mTorr) |

NA, not applicable

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1-15. (canceled)

16. The stable pharmaceutical formulation of claim 27, wherein the antibody comprises a heavy chain comprising the amino acid sequence of residues 1-445 of SEQ ID NO: 9 and a light chain comprising the amino acid sequence of SEQ ID NO: 10.

17. The stable pharmaceutical formulation of claim 27, wherein the antibody comprises a heavy chain comprising the amino acid sequence of SEQ ID NO: 9 and a light chain comprising the amino acid sequence of SEQ ID NO: 10.

18-23. (canceled)

24. A stable liquid pharmaceutical formulation comprising, in an aqueous solution:
(i) a human antibody that specifically binds to human cytotoxic T-lymphocyte-associated protein 4 (hCTLA-4) at a concentration of from 5±0.5 mg/ml to 150±15 mg/ml, wherein the antibody comprises a HCVR comprising HCDR1, HCDR2, and HCDR3 domains comprising the amino acid sequences of SEQ ID NOs: 3, 4, and 5, respectively, and LCDR1, LCDR2, and LCDR3 domains comprising the amino acid sequences of SEQ ID NOs: 6, 7, and 8, respectively;
(ii) 5 mM to 25 mM histidine;
(iii) 3% w/v to 12% w/v sucrose; and
(iv) 0.05% w/v to 0.25% w/v polysorbate 20,
wherein the aqueous solution has a pH of from 5.8 to 6.2.

25. The stable pharmaceutical formulation of claim 24 that is a reconstituted formulation.

26. A stable lyophilized pharmaceutical formulation of an antibody that specifically binds to human cytotoxic T-lymphocyte-associated protein 4 (hCTLA-4), made by lyophilizing an aqueous solution comprising:
(i) a human antibody that specifically binds to human cytotoxic T-lymphocyte-associated protein 4 (hCTLA-4) at a concentration of from 5±0.5 mg/ml to 150±15 mg/ml, wherein the antibody comprises a HCVR comprising HCDR1, HCDR2, and HCDR3 domains comprising the amino acid sequences of SEQ ID NOs: 3, 4, and 5, respectively, and LCDR1, LCDR2, and LCDR3 domains comprising the amino acid sequences of SEQ ID NOs: 6, 7, and 8, respectively;
(ii) 5 mM to 25 mM histidine;
(iii) 3% w/v to 12% w/v sucrose; and
(iv) 0.05% w/v to 0.25% w/v polysorbate 20,
wherein the aqueous solution has a pH of from 5.8 to 6.2.

27. The stable pharmaceutical formulation of claim 24, wherein the antibody comprises a heavy chain variable region (HCVR) comprising the amino acid sequence of SEQ ID NO: 1, and a light chain variable region (LCVR) comprising the amino acid sequence of SEQ ID NO: 2.

28. The stable pharmaceutical formulation of claim 27, wherein the antibody (a) has a human IgG heavy chain constant region; (b) has a human heavy chain constant region of isotype IgG1; or (c) has a human heavy chain constant region of isotype IgG4.

29-33. (canceled)

34. The stable pharmaceutical formulation of claim 24, wherein the aqueous solution comprises: (i) about 50 mg/ml±5 mg/ml of the antibody; (ii) about 10 mM±1 mM histidine; (iii) about 5% w/v±0.5% w/v sucrose; and (iv) about 0.1%±0.01% w/v polysorbate 20.

35. The stable pharmaceutical formulation of claim 24, wherein the aqueous solution comprises: (i) about 100 mg/ml±10 mg/ml of the antibody; (ii) about 20 mM±2 mM histidine; (iii) about 10% w/v±1% w/v sucrose; and (iv) about 0.2%±0.02% w/v polysorbate 20.

36. (canceled)

37. The stable pharmaceutical formulation of claim 24, wherein:
(a) at least 90% of the native form of the antibody is recovered after 24 months of storage at 5° C., as determined by size exclusion-ultra performance liquid chromatography (SE-UPLC);
(b) at least 95% of the native form of the antibody is recovered after 24 months of storage at 5° C., as determined by SE-UPLC; or
(c) at least 97% of the native form of the antibody is recovered after 24 months of storage at 5° C., as determined by SE-UPLC.

38-39. (canceled)

40. The stable pharmaceutical formulation of claim 24, wherein:
(a) at least 90% of the native form of the antibody is recovered after six months of storage at 25° C. and 60% relative humidity, as determined by SE-UPLC;
(b) at least 96% of the native form of the antibody is recovered after six months of storage at 25° C. and 60% relative humidity, as determined by SE-UPLC;
(c) at least 90% of the native form of the antibody is recovered after one month of storage at 45° C. or 50° C., as determined by SE-UPLC;
(d) at least 95% of the native form of the antibody is recovered after one month of storage at 45° C. or 50° C., as determined by SE-UPLC;
(e) at least 90% of the native form of the antibody is recovered after sixty minutes of agitation at ambient temperature, as determined by SE-UPLC; or
(f) at least 96% of the native form of the antibody is recovered after sixty minutes of agitation at ambient temperature, as determined by SE-UPLC.

41-45. (canceled)

46. The stable pharmaceutical formulation of claim 24, wherein:
(a) the formulation comprises no more than 2% high molecular weight (HMW) species after 24 months of storage at 5° C., as determined by SE-UPLC;
(b) the formulation comprises no more than 2.5% HMW species after six months of storage at 25° C. and 60% relative humidity, as determined by SE-UPLC;
(c) the formulation comprises no more than 2% high molecular weight (HMW) species after one month of storage at 45° C., as determined by SE-UPLC;
(d) the formulation comprises no more than 3% high molecular weight (HMW) species after one month of storage at 50° C., as determined by SE-UPLC; or
(e) the formulation comprises no more than 2% high molecular weight (HMW) species after sixty minutes of agitation at ambient temperature, as determined by SE-UPLC.

47-50. (canceled)

51. The stable pharmaceutical formulation of claim 24, contained in a glass vial, in a syringe, or in a large volume device or bolus injector.

52. (canceled)

53. The stable pharmaceutical formulation of claim 51, wherein (a) the syringe comprises a fluorocarbon-coated plunger, (b) the syringe is a low tungsten syringe, (c) the syringe is a prefilled syringe, or (d) the syringe is a prefilled staked needle syringe.

54-56. (canceled)

57. A pen or autoinjector delivery device containing a stable pharmaceutical formulation of claim 24.

58. The delivery device of claim 57 that is (a) a disposable pen delivery device, or (b) a reusable pen delivery device.

59. (canceled)

60. A container containing a stable pharmaceutical formulation of claim 24.

61. A kit comprising (i) a container containing the stable pharmaceutical formulation of claim 24, and (ii) labeling for use of the pharmaceutical formulation.

62. The kit of claim 61, wherein the labeling recites (a) subcutaneous administration of the pharmaceutical formulation, or (b) intravenous administration of the pharmaceutical formulation.

63. (canceled)

64. A unit dosage form comprising a stable pharmaceutical formulation of claim 24, wherein the anti-CTLA-4 antibody is present in an amount of from 1 mg to 500 mg.

65. The unit dosage form of claim 64, wherein the formulation is fa) contained in a glass vial, (b) contained in a syringe, or (c) contained in a prefilled syringe.

66-67. (canceled)

68. A safety system delivery device containing a stable pharmaceutical formulation of claim 24.

69. The safety system delivery device of claim 68, wherein the device includes (a) a safety sleeve configured to extend by manual operation, or (b) a safety sleeve configured to automatically extend following injection of the stable pharmaceutical formulation.

70. (canceled)
